(19) United States
(12) Patent Application Publication
(10) Pub. No.: US 2021/0260070 A1
(43) Pub. Date: Aug. 26, 2021

(54) LYSIN CF-301 RESENSITIZES METHICILLIN-RESISTANT STAPHYLOCOCCUS AUREUA (MRSA) TO PENICILLIN DERIVATIVES AND FIRST GENERATION CEPHALOSPORINS

(71) Applicant: CONTRAFECT CORPORATION, Yonkers, NY (US)

(72) Inventor: Raymond SCHUCH, Mountain Lakes, NJ (US)

(21) Appl. No.: 17/252,998

(22) PCT Filed: Jun. 21, 2019
(86) PCT No.: PCT/US2019/038525

§ 371 Date: Dec. 16, 2020

Related U.S. Application Data
(60) Provisional application No. 62/688,756, filed on Jun. 22, 2018.

(51) Int. Cl. A61K 31/546; A01N 37/46; A01N 43/90; A61K 31/431; A61P 31/04

(52) U.S. Cl. CPC A61K 31/546; A01N 37/46; A61P 31/04; A61K 31/431; A01N 43/90

(57) ABSTRACT

Disclosed are methods of resensitizing a Gram-positive bacterium in a subject to at least one β-lactam antibiotic, comprising co-administering the Gram-positive bacterium with the at least one β-lactam antibiotic and a lysin polypeptide, thereby resensitizing the Gram-positive bacterium in the subject to the at least one β-lactam antibiotic.

### CROSS-REFERENCE TO RELATED APPLICATIONS

This application claims the benefit of, and relies on the filing date of, U.S. provisional patent application No. 62/688,756, filed 22 Jun. 2018, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Jun. 18, 2019, is named 0341_0017_00_304.txt and is 36,864 bytes in size.

### FIELD OF THE DISCLOSURE

The present disclosure relates generally to antibacterial agents and more specifically to lysin polypeptides and the use of these peptides in combination with antibiotics to kill Gram-positive bacteria and resensitize Gram-positive bacteria to antibiotics.

### BACKGROUND OF THE INVENTION

Antibiotic resistance is on the increase worldwide, influenced, inter alia, by (a) increased and prolonged use of antibiotics administered to treat a variety of illnesses and other conditions; (b) poor patient compliance; and (c) a paucity of new antimicrobial agents that can be deployed against pathogens that have developed resistance to existing antibiotics.

Bacteriophage endolysins (lysins) represent a promising alternative or complementary approach to combating bacterial infections and to overcoming bacterial resistance. Lysins are peptidoglycan hydrolases that can be produced naturally by bacteriophages. When contacting the bacteria from the outside, recombinantly-produced lysin polypeptides directly lyse and kill the bacteria [1], [2]. Lysins may also overcome antibiotic resistance by facilitating access of the antibiotic agents to pathogens. Several studies have recently demonstrated the strong potential of these enzymes in human and veterinary medicine to control pathogens on mucosal surfaces, in organ-confined infections, and in systemic infections.

Gram-positive bacteria are surrounded by a cell wall containing polypeptides and polysaccharides. The Gram-positive cell wall appears as a broad, dense wall that may be about 20-80 nm thick and contains numerous interconnecting layers of peptidoglycan. Between 60% and 90% of the Gram-positive cell wall is peptidoglycan, providing cell shape, a rigid structure, and resistance to osmotic shock. The cell wall does not exclude the Gram stain crystal violet, allowing cells to be stained purple, and therefore classified as “Gram-positive.”

Bacteriophage lytic enzymes have been established as useful in the specific treatment of various types of infection in subjects through various routes of administration. See e.g., U.S. Pat. Nos. 5,985,271; 6,017,528; 6,056,955; 6,248,324; 6,254,866; and 6,264,945. U.S. Pat. No. 9,034,322 to Fischetti et al., which is hereby incorporated by reference in its entirety, is directed to bacteriophage lysins derived from Streptococcus suis bacteria, including the lysin PlySs2. These lysin polypeptides demonstrate broad killing activity against multiple bacteria, including Gram-positive bacteria such as Staphylococcus, Streptococcus Group B, Enterococcus, and Listeria bacterial strains.

The PlySs2 lysin is capable of killing Staphylococcus aureus bacteria in animal models and synergizing with antibiotics. PlySs2 was shown to be effective against antibiotic-resistant Staphylococcus aureus, such as methicillin-resistant Staphylococcus aureus (MRSA) and vancomycin-resistant Staphylococcus aureus (VRSA).

Although antimicrobial resistance is a well-recognized global health threat, with respect to β-lactam antibiotics, strategies to overcome resistance have been limited to the use of higher doses of β-lactam antibiotics, combinations with β-lactamase inhibitors, and development of new classes of antibiotics. Emerging resistance to drug classes used to treat MRSA (e.g. glycopeptides, cyclic lipopeptide, and oxazolidinones) represents a new threat. PlySs2 and other Gram-positive lysins are a new class of recombinantly-produced, bacteriophage-derived lysins (cell wall hydrolases) developed for the treatment, for example, of S. aureus infective endocarditis and bacteremia used in addition to standard-of-care antibiotics.

PlySs2 demonstrates: 1) rapid and potent bacteriolytic effects against all S. aureus strains including MRSA and vancomycin-, daptomycin- and linezolid-resistant strains; 2) potent antibiofilm activity; 3) synergy with antistaphylococcal antibiotics 4) low propensity for bacterial resistance; and 5) the ability to suppress the emergence of resistance to antibiotics in vitro and in vivo.

The ability of PlySs2 and other Gram-positive lysins to resensitize drug resistant bacteria to β-lactam antibiotics that were previously inactive and thereby restore the utility of the β-lactam antibiotics, would thus be beneficial.

### SUMMARY OF THE INVENTION

This application discloses the use of lysin polypeptides in a method of resensitizing a Gram-positive bacterium to at least one β-lactam antibiotic. In one aspect, the method comprises co-administering to a subject at least one β-lactam antibiotic and a lysin polypeptide, thereby resensitizing the Gram-positive bacterium in the subject to the at least one β-lactam antibiotic. In certain embodiments, the method further comprises after the co-administering step, a step of administering the at least one β-lactam antibiotic to the subject in an amount effective to reduce the population, kill, inhibit the growth, and/or eradicate the resensitized Gram-positive bacterium.

In another aspect, the method comprises co-administering to a non-living surface at least one β-lactam antibiotic and a lysin polypeptide, wherein the non-living surface is infected with a Gram-positive bacterium that is resistant to the at least one β-lactam antibiotic and wherein the co-administration step reduces the amount of Gram-positive bacterium on the non-living surface and resensitizes the Gram-positive bacterium to the at least one β-lactam antibiotic. In certain embodiments, the method further comprises after the co-administering step, a step of administering the at least one β-lactam antibiotic to the non-living surface in an amount effective to reduce the population, kill, inhibit the growth, and/or eradicate the resensitized Gram-positive bacterium. In certain embodiments, the non-living surface is a medical device, including but not limited to, a catheter, an inhaler, intubation device, a valve, surgical instrument, or prosthesis.

In certain embodiments, the lysin polypeptide is administered prior to the at least one β-lactam antibiotic, such as at least 24 hours prior to the at least one β-lactam antibiotic. In certain embodiments, the lysin polypeptide and the at least one β-lactam antibiotic are administered substantially simultaneously. In certain embodiments, the lysin polypeptide is administered in a single dose. In certain embodiments, the at least one β-lactam antibiotic is not effective to reduce the population, kill, inhibit the growth, and/or eradicate the Gram-positive bacterium before administration of the lysin polypeptide.

In certain embodiments of the methods for resensitizing a Gram-positive bacterium disclosed herein, the Gram-positive bacterium is a Staphylococcus bacterium, such as Staphylococcus aureus. In certain embodiments, the at least one β-lactam antibiotic is selected from the group consisting of oxacillin, nafcillin, and cefazolin. In certain embodiments, the at least one β-lactam antibiotic is oxacillin. In certain embodiments, the Gram-positive bacteria is MRSA, and in some embodiments, the Gram-positive bacteria is VRSA.

In certain aspects of the disclosure, the Gram-positive bacterium causes skin or soft tissue infection, bacteremia, endocarditis, bone infections such as osteomyelitis, joint infections, and/or pneumonia. In certain aspects, after administration of the lysin polypeptide, the at least one β-lactam antibiotic is effective at a dosage below its MIC dose to reduce the population, kill, inhibit the growth, and/or eradicate the Gram-positive bacterium. In certain aspects, the lysin polypeptide is effective at a dosage below its MIC dose to resensitize the Gram-positive bacterium. In certain embodiments, both the lysin polypeptide and the at least one (3-lactam antibiotic, when administered either sequentially or simultaneously, are effective to reduce the population, kill, inhibit the growth, and/or eradicate the Gram-positive bacterium at doses below their MIC dose.

In certain embodiments, the lysin polypeptide comprises an amino acid sequence selected from the group consisting of SEQ ID NOs. 1-17 or variants thereof having at least 80% amino acid identity to SEQ ID NOs. 1-17 and lytic activity. In certain embodiments, the lysin polypeptide comprises an amino acid sequence of SEQ ID NO: 1. In certain embodiments, the lysin polypeptide comprises an amino acid sequence selected from the group consisting of SEQ ID NOs. 3-17.

### BRIEF DESCRIPTION OF THE FIGURES

FIG. 1 is a graph depicting the fold change in oxacillin and PlySs2 lysin MIC values as a function of days of serial passage under resistance conditions for MRSA strain MW2 in a first trial, as described in Example 2.

FIG. 2 is a graph depicting the fold change in oxacillin and PlySs2 lysin MIC values as a function of days of serial passage under resistance conditions for MRSA strain MW2 in a second trial, as described in Example 2.

FIG. 3 is a graph depicting the fold change in oxacillin and PlySs2 lysin MIC values as a function of days of serial passage under resistance conditions for MRSA strain MW2 in a third trial, as described in Example 2.

### DETAILED DESCRIPTION

### Definitions

As used herein, the following terms and cognates thereof shall have the following meanings unless the context clearly indicates otherwise:

“Carrier,” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition.

“Bactericidal” refers to the property of causing the death of bacteria or capable of killing bacteria to an extent of at least a 3-log 10 (99.9%) or better reduction among an initial population of bacteria over an 18-24 hour period.

“Bacteriostatic” refer to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity, or DNA or protein synthesis in bacteria. Nonlimiting examples of antibiotics active against Gram-positive bacteria include methicillin, vancomycin, daptomycin, mupirocin, lysostaphin, penicillins, cloxacillin, erythromycin, carbapenems, cephalosporins, glycopeptides, lincosamides, azithromycin, clarithromycin, roxithromycin, telithromycin, spiramycin, and fidaxomicin.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multidrug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” is intended to embrace separate administration of two agents, such as a lysin peptide and an antibiotic or any other antibacterial agent in a sequential manner as well as administration of these agents in a substantially simultaneous manner, such as in a single mixture/composition or in doses given separately, but nonetheless administered substantially simultaneously to the subject, for example at different times in the same day or 24-hour period. Such co-administration of lysin peptides with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, the lysin polypeptide could be administered only initially within 24 hours of the first antibiotic use, and then the antibiotic use may continue without further administration of the lysin polypeptide.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive or Gram-negative bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-positive bacteria, whether such infection be systemic, topical or otherwise concentrated or confined to a particular organ or tissue.

“Polypeptide” is used interchangeably with the terms “protein” and “peptide,” and refers to a polymer made from amino acid residues. In certain embodiments, the polypeptide has at least about 30 amino acid residues. The term may include not only polypeptides in isolated form, but also active fragments and derivatives thereof. The term “polypeptide” also encompasses fusion proteins or fusion polypeptides comprising a modified lysin polypeptide and maintaining the lysin function. Depending on context, a polypeptide can be a naturally-occurring polypeptide or a recombinant, engineered, or synthetically-produced polypeptide. A particular lysin polypeptide can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining lytic activity against the same or at least one common target bacterium.

“Fusion polypeptide” refers to an expression product resulting from the fusion of two or more nucleic acid segments, resulting in a fused expression product typically having two or more domains or segments with different properties or functionality. In certain embodiments, the term “fusion polypeptide” also refers to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” Thus, the open-ended expression “a polypeptide comprising” a certain structure includes larger molecules than the recited structure such as fusion polypeptides or constructs. The constructs referred to herein can be made as fusion polypeptides or as conjugates (by linking two or more moieties).

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a modified lysin polypeptide and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more lysin polypeptides or active fragments thereof. These can be used to make a fusion polypeptide with lytic activity.

“Active fragment” refers to a portion of a polypeptide that retains one or more functions or biological activities of the isolated polypeptide from which the fragment was taken. As used herein, an active fragment of a lysin polypeptide inhibits the growth, or reduces the population, or kills at least one Gram-positive bacterial species, such as S. aureus.

“Amphipathic peptide” refers to a peptide having both hydrophilic and hydrophobic functional groups. In certain embodiments, secondary structure places hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides which are synthetically produced peptides composed of mostly positively charged amino acid residues, such as lysine and/or arginine residues. The amino acid residues that are not positively charged can be neutrally charged amino acid residues, negatively charged amino acid residues, and/or hydrophobic amino acid residues.

“Hydrophobic group” refers to a chemical group such as an amino acid side chain which has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Be), proline (Pro), phenylalanine (Phe), methionine (Met), and tryptophan (Trp).

“Augmenting” as used herein refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a subthreshold level, i.e., a level at which if the active substance is employed individually produces no or a very limited effect. The effect can be measured by assays such as a checkerboard assay, described here.

“Treatment” refers to any process, action, application, therapy, or the like, wherein a subject, including a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of the bacteria in the subject and thereby controlling or reducing a bacterial infection in a subject or bacterial contamination of an organ, tissue, or environment. Thus “treatment” that reduces incidence is effective to inhibit growth of at least one Gram-positive bacterium in a particular milieu, whether it be a subject or an environment. On the other hand, “treatment” of an already established infection refers to reducing the population, killing, inhibiting the growth, and/or eradicating the Gram-positive bacteria responsible for an infection or contamination.

The term “preventing” and includes the prevention of the incidence, recurrence, spread, onset, or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting it is reduced, and such constitute examples of prevention. With specific reference to biofilm prevention, the term includes prevention of the formation of biofilm, for example by interfering with the adherence of bacteria on a surface of interest, such as the surface of a medical device (e.g., inhaler, catheter, intubation, valve, or other prosthesis).

“Contracted disease” refers to a disease manifesting with clinical or subclinical symptoms, such as the detection of fever, sepsis, or bacteremia, as well as disease that may be detected by growth of a bacterial pathogen (e.g., in culture) when symptoms associated with such pathology are not yet manifest. With respect to medical devices, in particular, a contracted disease shall include a biofilm containing bacteria, such as Staphylococcus or Streptococcus bacteria, and forming when such a device is in use.

The term “derivative” in the context of a peptide or polypeptide (which as stated herein includes an active fragment) is intended to encompass, for example, a polypeptide modified to contain one or more chemical moieties other than an amino acid that do not substantially adversely impact or destroy the polypeptides's activity, such as lytic activity. The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to lysin polypeptides include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a lysin polypeptide or fused to a lysin polypeptide without interfering with normal functions of cellular proteins. In certain embodiments, a polynucleotide encoding a fluorescent protein is inserted upstream or downstream of the lysin polynucleotide sequence. This will produce a fusion protein (e.g., Lysin Polypeptide::GFP) that does not interfere with cellular function or function of a lysin polypeptide to which it is attached. Polyethylene glycol (PEG) conjugation to proteins has been used as a method for extending the circulating half-life of many pharmaceutical proteins. Thus, in the context of lysin polypeptide derivatives, the term “derivative” encompasses lysin polypeptides chemically modified by covalent attachment of one or more PEG molecules. It is anticipated that pegylated lysin polypeptides will exhibit prolonged circulation half-life compared to the unpegylated lysin polypeptides, while retaining biological and therapeutic activity. Another example is the use of “artilysins”, whereby a short polycationic and amphipathic alpha helices are appended to the N- or C-termini of a lysin polypeptide to improve in vitro antibacterial activity, such as a streptococcal lysin to improve in vitro anti-streptococcal activity.

“Percent amino acid sequence identity” refers to the percentage of amino acid residues in a candidate sequence that are identical with the amino acid residues in the reference polypeptide sequence, such as a lysin polypeptide sequence, after aligning the sequences and introducing gaps, if necessary, to achieve the maximum percent sequence identity, and not considering any conservative substitutions as a part of the sequence identity. Alignment for purposes of determining percent amino acid sequence identity can be achieved in various ways that are within the skill in the art, for example, using publicly available software such as BLAST or software available commercially for example from DNASTAR. Two or more polypeptide sequences can be anywhere from 0-100% identical, or any integer value there between. In the context of the present disclosure, two polypeptides are “substantially identical” when at least 80% of the amino acid residues (preferably at least about 85%, at least about 90%, and preferably at least about 95%, at least about 98%, or at least 99%) are identical. The term “percent (%) amino acid sequence identity” as described herein applies to peptides as well. Thus, the term “Substantially identical” will encompass mutated, truncated, fused, or otherwise sequence-modified variants of isolated polypeptides and peptides, such as those described herein, and active fragments thereof, as well as polypeptides with substantial sequence identity (e.g., at least 80%, at least 85%, at least 90%, at least 95% identity, at least 98% identity, or at least 99% identity as measured for example by one or more methods referenced above) as compared to the reference (wild type or other intact) polypeptide. Two amino acid sequences are “substantially homologous” when at least about 80% of the amino acid residues (preferably at least about 85%, at least about 90%, at least about 95%, at least about 98% identity, or at least about 99% identity) are identical, or represent conservative substitutions. The sequences of polypeptides of the present disclosure, are substantially homologous when one or more, or several, or up to 10%, or up to 15%, or up to 20% of the amino acids of the polypeptide, such as the lysin and/or fusion polypeptides described herein, are substituted with a similar or conservative amino acid substitution, and wherein the resulting polypeptide, such as the lysin and/or fusion polypeptides described herein, have at least one activity, antibacterial effects, and/or bacterial specificities of the reference polypeptide, such as the lysin and/or fusion polypeptides described herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides. In certain embodiments, the biofilm may contain Staphylococcus and/or Streptococcus bacteria.

“Suitable” in the context of an antibiotic being suitable for use against certain bacteria refers to an antibiotic that was found to be effective against those bacteria even if resistance subsequently developed.

“Wild-type PlySs2 lysin” and “PlySs2 lysin,” refer to a polypeptide having the amino acid sequence: MTTVNEALNNVRAQVGSGVSVGNGECYALASWYERMISPDATVGLGAGVGWVSGAI GDTISAKNIGSSYNWQANGWTVSTSGPFKAGQIVTLGATPGNPYGHVVIVEAVDGDRL TILEQNYGGKRYPVRNYYSAASYRQQVVHYITPPGTVAQSAPNLAGSRSYRETGTMTV TVDALNVRRAPNTSGEIVAVYKRGESFDYDTVIIDVNGYVWVSYIGGSGKRNYVATG ATKDGKRFGNAWGTFK (SEQ ID NO: 1; 245 amino acid residues including the initial methionine residue which is removed during post-translational processing, leaving a 244-amino acid peptide).

“Modified lysin polypeptide” as used herein refers to a non-naturally occurring variant (or active fragment thereof) of the wild-type PlySs2 lysin. The modified lysin polypeptide has at least one amino acid substitution in the CHAP domain and/or the SH3b domain, and inhibits the growth, reduces the population, or kills at least one species of Gram-positive bacteria, such as S. aureus. Modified lysin polypeptides, such as modified lysin polypeptides having the amino acid sequence selected from the group consisting of SEQ ID NOs: 3-17, are disclosed, for example, in PCT Application No. PCT/US2019/019638, incorporated in its entirety by reference herein. As the term is used herein, lysin polypeptides encompass modified lysin polypeptides.

“Substantially” used in the context of lytic activity (antimicrobial activity) of a lysin polypeptide or fragment thereof of the present disclosure means at least a considerable portion of the antibacterial activity of the wild-type PlySs2 lysin, such that, on the basis of such activity, the lysin polypeptide or fragment thereof would be useful alone or together with other antimicrobial agents, such as one or more antibiotics and/or lysostaphin, to inhibit, combat, or eliminate Staphylococcal or Streptococcal bacterial infection by killing these bacteria. Nonlimiting examples of such substantial activity compared to the wild-type PlySs2 lysin include no more than about 5, such as no more than about 4, no more than about 3, or no more than about 2, times the MIC of the wild-type lysin. Other measures of activity can be, for example, minimum biofilm eliminating concentration (MBEC) or in vivo efficacy using, for example, an animal model, such as the mouse neutropenic thigh infection model (MNTI). Still other measures can be the ability to synergize with antibiotics (such as vancomycin, daptomycin, or β-lactam antibiotics, including oxacillin, nafcillin, and cefazolin) or the ability to ameliorate, prevent, or delay development of, bacterial resistance of antibiotics.

### Lysin Polypeptides

The present application relates to the use of lysin polypeptides in a method of resensitizing a Gram-positive bacterium to at least one β-lactam antibiotic.

Lysin polypeptides, including the lysin PlySs2, demonstrate broad killing activity against multiple bacteria, particularly Gram-positive bacteria, including Staphylococcus and Streptococcus bacterial strains, provide remarkable synergy in combination with certain antibiotics including β-lactam antibiotics, and can significantly reduce the effective MIC doses required for the antibiotics. Furthermore, lysin polypeptides, including the lysin PlySs2, provide the ability to resensitize certain β-lactam antibiotics to Gram-positive bacterial strains which were not previously susceptible to the β-lactam antibiotics.

The lysin polypeptides may be combined or co-administered with antibiotics, including, for example, β-lactam antibiotics such as one or more of oxacillin, nafcillin, cefazolin and/or similar antibiotics, in particular, for use in resensitizing a Gram-positive bacteria that has developed resistance to the antibiotic. In a particular aspect, a lysin polypeptide is combined or co-administered with oxacillin to resensitize a Gram-positive bacteria, including S. aureus, particularly including MRSA, to oxacillin. In a particular aspect, a lysin polypeptide is combined or co-administered with nafcillin to resensitize a Gram-positive bacteria, including S. aureus, particularly including MRSA, to nafcillin. In a particular aspect, a lysin polypeptide is combined or co-administered with cefazolin to resensitize a Gram-positive bacteria, including S. aureus, particularly including MRSA, to cafazolin. In an aspect of the invention, combination or co-administration with a lysin polypeptide significantly reduces the dose of antibiotic required to kill a Gram-positive bacteria, such as S. aureus, particularly including MRSA.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-IIII [J. E. Celis, ed. (1994)]; “Current Protocols in Immunology” Volumes I-III [Coligan, J. E., ed. (1994)]; “Oligonucleotide Synthesis” [(M. J. Gait ed. 1984)]; “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells and Enzymes” [IRL Press, (1986)]; and B. Perbal, “A Practical Guide To Molecular Cloning” (1984).

Further disclosed herein is a lysin-dependent enhancement of antibiotic efficacy in Gram-positive bacterial infections under conditions wherein the use of an antibiotic in the absence of the lysin fails. Data are presented herein illustrating PlySs2-mediated enhancement of antibiotic activity and indicating a general synergy between lysins and β-lactam antibiotics, as well as resensitization of the Gram-positive bacteria to the β-lactam antibiotics.

The lysin polypeptides disclosed herein, including PlySs2 and modified lysin polypeptides, are capable of killing numerous distinct strains and species of Gram-positive bacteria, including Staphylococcal, Streptococcal, Listeria, or Enterococcal bacteria. In particular, PlySs2 is active in killing Staphylococcus strains, including both antibiotic-sensitive and antibiotic-resistant Staphylococcus aureus strains (e.g., MSSA and MRSA). PlySs2 and modified lysin polypeptides may also be active in killing Streptococcus strains, including Group A and Group B streptococcus strains.

In some embodiments, the present lysin polypeptides reduce the minimum inhibitory concentration (MIC) of an antibiotic. Any known method to assess MIC may be used. In some embodiments, a checkerboard assay is used to determine the effect of a lysin on antibiotic concentration. The checkerboard assay is based on a modification of the CLSI method for MIC determination by broth microdilution (See Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, Pa., which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of lysin diluted e.g., 2-fold along the vertical axis. The lysin and antibiotic dilutions are then combined, so that each column has a constant amount of antibiotic and doubling dilutions of lysin, while each row has a constant amount of lysin and doubling dilutions of antibiotic. Each well thus has a unique combination of lysin and antibiotic. Bacteria are added to the drug combinations at a given concentration. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣFICmin) is used to determine the effect of the lysin/antibiotic combination.

In certain embodiments, the lysin polypeptide is PlySs2 or an active fragment thereof. PlySs2 is a bacteriophage lysin that may be derived from Streptococcus suis bacteria. PlySs2 demonstrates broad killing activity against multiple bacteria, including Gram-positive bacteria, including Staphylococcus, Streptococcus, Enterococcus, and Listeria bacterial strains, including antibiotic-resistant Staphylococcus aureus, such as MRSA and VRSA. Wild-type PlySs2 has the following amino acid sequence: MTTVNEALNNVRAQVGSGVSVGNGECYALASWYERMISPDATVGLGAGVGWVSGAIG DTISAKNIGSSYNWQANGWTVSTSGPFKAGQIVTLGATPGNPYGHVVIVEAVDGDRLTI LEQNYGGKRYPVRNYYSAASYRQQVVHYITPPGTVAQSAPNLAGSRSYRETGTMTVTV DALNVRRAPNTSGEIVAVYKRGESFDYDTVIIDVNGYVWVSYIGGSGKRNYVATGATK DGKRFGNAWGTFK (SEQ ID NO: 1). SEQ ID NO: 1 has 245 amino acid residues, including the initial methionine residue which is removed during post-translational processing, leaving a 244-amino acid polypeptide. Amino acid residues 1 to 146 correspond to the CHAP domain, and amino acid residues 157 to 245 correspond to the SH3b domain; the naturally occurring linker between the two domains is PPGTVAQSAP (SEQ ID NO: 2).

In certain embodiments, the lysin polypeptide is a modified lysin polypeptide having lytic activity. As used herein, “lytic activity” encompasses the ability of a lysin to kill bacteria, reduce the population of bacteria or inhibit bacterial growth. Lytic activity also encompasses the ability to remove or reduce a biofilm and/or the ability to reduce the minimum inhibitory concentration (MIC) of an antibiotic. A modified lysin polypeptide may comprise at least one amino acid substitution as compared to a wild-type PlySs2 lysin polypeptide, wherein the wild-type PlySs2 lysin polypeptide has an amino acid sequence of SEQ ID NO: 1, a cysteine, histidine-dependent amidohydrolase/peptidase (CHAP) domain, and a cell wall binding (SH3b) domain, and wherein the at least one amino acid substitution is in the CHAP domain and/or the SH3b domain, wherein the modified lysin polypeptide inhibits the growth, reduces the population, or kills at least one species of Gram-positive bacteria. Typically, the modified lysin polypeptide has reduced immunogenicity as compared to the wild-type PlySs2 (SEQ ID NO: 1). In certain embodiments, the at least one amino acid substitution is in the CHAP domain. In certain embodiments, the at least one amino acid substitution is in the SH3b domain. In certain embodiments, the at least one amino acid substitution is in the CHAP domain and the SH3b domain.

In some embodiments, the modified lysin polypeptide has at least 80%, such as at least 85%, such as at least 90%, such as at least 95%, such as at least 98% or such as at least 99% sequence identity with a reference lysin polypeptide, such as wild-type PlySs2 (SEQ ID NO: 1).

In some embodiments, the modified lysin polypeptide retain one or more functional or biological activities of the reference lysin polypeptide. In some embodiments, the modification improves the antibacterial activity of the lysin. Typically, the lysin variant has improved in vitro antibacterial activity (e.g., in buffer and/or media) in comparison to the reference lysin polypeptide. In other embodiments, the lysin variant has improved in vivo antibacterial activity (e.g., in an animal infection model).

In certain embodiments, the at least one substitution is in the CHAP domain in at least one position selected from amino acid residue 35, 92, 104, 128, and 137 of SEQ ID NO: 1. In certain embodiments, the at least one substitution is in the SH3b domain in at least one position selected from amino acid residue 164, 184, 195, 198, 204, 206, 212, and 214 of SEQ ID NO: 1. In certain embodiments, modified lysin polypeptide has at least one substitution in the CHAP domain in at least one position selected from amino acid 35, 92, 104, 128, and 137 of SEQ ID NO: 1 and at least one substitution in the SH3b domain in at least one position selected from amino acid 164, 184, 195, 198, 204, 206, 212, and 214 of SEQ ID NO: 1.

In some embodiments, the at least one amino acid substitution in the CHAP domain is selected from the group consisting of R35E, L92W, V104S, V128T and Y137S. In certain embodiments, the at least one amino acid substitution in the SH3b domain is selected from the group consisting of Y164N, Y164K, N184D, R195E, S198H, S198Q, V204K, V204A, I206E, V212A, V212E, and V214G.

In certain embodiments, the modified lysin polypeptide has at least one amino acid substitution in the CHAP domain selected from the group consisting of R35E, L92W, V104S, V128T and Y137S and at least one amino acid substitution in the SH3b domain selected from the group consisting of Y164N, Y164K, N184D, R195E, S198H, S198Q, V204K, V204A, I206E, V212A, V212E, and V214G.

In yet other embodiments, the modified lysin polypeptide has at least two amino acid substitutions in the CHAP domain; in still other embodiments, the modified lysin polypeptide has at least two amino acid substitutions in the SH3b domain; in other embodiments, the modified lysin polypeptide has at least three amino acid substitutions in the SH3b domain. In yet other embodiments, the modified lysin polypeptide has 5, 6, 7, or 8 amino acid substitutions distributed between the CHAP and SH3b domains, and in certain embodiments, the amino acid sequence of SEQ ID NO: 1 is modified by 3-9 of the amino acid substitutions selected from the group consisting of: R35E, L92W, V104S, V128T, Y137S, Y164N, Y164K, N184D, R195E, S198H, S198Q, V204K, V204A, 1206E, V212E, V212A, and V214G.

In certain embodiments, the modified lysin polypeptide comprises the following amino acid substitutions relative to the amino acid sequence of SEQ ID NO: 1: (i) L92W, V104S, V128T, and Y137S (pp55); (ii) Y164N, N184D, R195E, V204K, and V212E (pp388); (iii) L92W, V104S, V128T, Y137S, S198H, and I206E (pp61); (iv) L92W, V104S, V128T, Y137S, S198Q, V204A, and V212A (pp65); (v) L92W, V104S, V128T, Y137S, Y164K, N184D, and S198Q (pp296); (vi) V128T, Y137S, and Y164K (pp616); (vii) R35E, L92W, V104S, V128T, and Y137S (pp400); (viii) L92W, V104S, V128T, Y137S, Y164K, V204K, and V212E (pp628); (ix) L92W, V104S, V128T, Y137S, Y164K, N184D, S198Q, V204K, and V212E (pp632); (x) L92W, V104S, V128T, Y137S, Y164N, and N184D (pp324); (xi) L92W, V104S, V128T, Y137S, Y164N, and R195E (pp325); (xii) L92W, V104S, V128T, Y137S, N184D, V204A, and V212A (pp341); (xiii) L92W, V104S, V128T, Y137S, and Y164K (pp619); (xiv) L92W, V104S, V128T, Y137S, Y164K, I206E, and V214G (pp642); and (xv) L92W, V104S, V128T, Y137S, N184D, and S198H (pp338). In certain embodiments, the modified lysin polypeptide has an amino acid sequence selected from one of SEQ ID NOs. 3-17.

In certain embodiments, the modified lysin polypeptide has at least 80% sequence identity with SEQ ID NO: 3, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 3. In certain embodiments, the encoded modified lysin polypeptide has at least 85%, 90%, 95%, 98%, or 99% sequence identity with SEQ ID NO: 3.

In certain embodiments, the modified lysin polypeptide has at least 80% sequence identity with SEQ ID NO: 4, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 4.

In certain embodiments, the modified lysin polypeptide has at least 80% sequence identity with SEQ ID NO: 5, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 5.

In certain embodiments, the modified lysin polypeptide has at least 80% sequence identity with SEQ ID NO: 6 wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 6.

In certain embodiments, the modified lysin polypeptide has at least 80% sequence identity with SEQ ID NO: 7, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 7.

In certain embodiments, the modified lysin polypeptide has at least 80% sequence identity with SEQ ID NO: 8, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 8.

In certain embodiments, the modified lysin polypeptide has at least 80% sequence identity with SEQ ID NO: 9, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 9.

In certain embodiments, the modified lysin polypeptide has at least 80% sequence identity with SEQ ID NO: 10, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 10.

In certain embodiments, the modified lysin polypeptide has at least 80% sequence identity with SEQ ID NO: 11, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 11.

In certain embodiments, the modified lysin polypeptide has at least 80% sequence identity with SEQ ID NO: 12, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 12.

In certain embodiments, the modified lysin polypeptide has at least 80% sequence identity with SEQ ID NO: 13, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 13.

In certain embodiments, the modified lysin polypeptide has at least 80% sequence identity with SEQ ID NO: 14, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 14.

In certain embodiments, the modified lysin polypeptide has at least 80% sequence identity with SEQ ID NO: 15, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 15.

In certain embodiments, the modified lysin polypeptide has at least 80% sequence identity with SEQ ID NO: 16, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 16.

In certain embodiments, the modified lysin polypeptide has at least 80% sequence identity with SEQ ID NO: 17, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 17.

In certain embodiments the modified lysin polypeptide comprises the following amino acid substitutions relative to the amino acid sequence of SEQ ID NO: 1: L92W, V104S, V128T, and Y137S. In certain embodiments the modified lysin polypeptide comprises the following amino acid substitutions relative to the amino acid sequence of SEQ ID NO: 1: L92W, V104S, V128T, Y137S, Y164K, N184D, and S198Q (pp296).

Also disclosed are active fragments of the modified lysin polypeptides disclosed herein, where the active fragments include one or more of the amino acid substitutions in the CHAP domain and/or the SH3b domain.

Further disclosed herein are chimeric lysins comprising a modified PlySs2 CHAP domain, as disclosed herein, and the binding domain of another lysin or the catalytic domain of another lysin and a modified PlySs2 SH3b domain, as disclosed herein.

Polynucleotides

In one aspect, the present disclosure is directed to an isolated polynucleotide comprising a nucleic acid molecule encoding a lysin polypeptide or active fragment thereof as disclosed herein. In certain embodiments, the lysin polypeptide is a PlySs2 lysin polypeptide (SEQ ID NO: 1). In certain embodiments, the lysin polypeptide is a selected from the group consisting of modified lysin polypeptides (SEQ ID NOs. 3-17). In certain embodiments, the encoded lysin polypeptide or active fragment thereof inhibits the growth, reduces the population, or kills at least one species of Gram-positive bacteria.

In certain embodiments, the nucleic acid molecule encodes a modified lysin polypeptide, wherein the modified lysin polypeptide comprises at least one amino acid substitution as compared to the wild-type PlySs2 polypeptide (SEQ ID NO: 1), wherein the modified lysin polypeptide comprises at least one amino acid substitution in the CHAP domain in at least one position selected from amino acid residue 35, 92, 104, 128, and 137 of SEQ ID NO: 1 and/or at least one amino acid substitution in the SH3b domain in at least one position selected from amino acid residue 164, 184, 195, 198, 204, 206, 212, and 214 of SEQ ID NO: 1. In certain embodiments, the nucleic acid molecule encodes a modified lysin polypeptide, wherein the modified lysin polypeptide comprises an amino acid substitution in amino acid residues of 92, 104, 128, and 137 of SEQ ID NO: 1. In certain embodiments, the nucleic acid molecule encodes a modified lysin polypeptide, wherein the modified lysin polypeptide comprises an amino acid substitution in amino acid residues 92, 104, 128, 137, 164, 184, and 198 of SEQ ID NO: 1.

In certain embodiments, the nucleic acid molecule encodes a modified lysin polypeptide, wherein the modified lysin polypeptide comprises one or more of the following amino acid substitutions relative to SEQ ID NO: 1: R35E, L92W, V104S, V128T, Y137S, Y164N, Y164K, N184D, R195E, S198H, S198Q, V204K, V204A, 1206E, V212E, V212A, and V214G. In certain embodiments, the nucleic acid molecule encodes a modified lysin polypeptide, wherein the modified lysin polypeptide comprises one or more of the following amino acid substitutions located in the CHAP domain: R35E, L92W, V104S, V128T and Y137S, and/or one or more of the following amino acid substitutions located in the SH3b domain: Y164N, Y164K, N184D, R195E, S198H, S198Q, V204K, V204A, I206E, V212A, V212E, and V214G.

In certain embodiments, the nucleic acid molecule encodes a modified lysin polypeptide, wherein the modified lysin polypeptide comprises the following amino acid substitutions relative to SEQ ID NO: 1: L92W, V104S, V128T, and Y137S. In certain embodiments, the nucleic acid molecule encodes a modified lysin polypeptide, wherein the modified lysin polypeptide comprises the amino acid sequence of SEQ ID NO: 3. In certain embodiments, the nucleic acid molecule encodes a modified lysin polypeptide having at least 80% sequence identity with SEQ ID NO: 3, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 90%, 95%, 98%, or 99% sequence identity with SEQ ID NO: 3.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, S198H, and I206E. In certain embodiments, the nucleic acid molecule encodes a modified lysin polypeptide, wherein the modified lysin polypeptide comprises the amino acid sequence of SEQ ID NO: 4. In certain embodiments, the nucleic acid molecule encodes a modified lysin polypeptide having at least 80%, 85%, 90%, 95%, 98%, or 99% sequence identity with SEQ ID NO: 4, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, S198Q, V204A, and V212A. In certain embodiments, the nucleic acid molecule encodes a modified lysin polypeptide, wherein the modified lysin polypeptide comprises the amino acid sequence of SEQ ID NO: 5. In certain embodiments, the nucleic acid molecule encodes a modified lysin polypeptide having at least 80%, 85%, 90%, 95%, 98%, or 99% sequence identity with SEQ ID NO: 5, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, N184D, and S198Q. In certain embodiments, the nucleic acid molecule encodes a modified lysin polypeptide, wherein the modified lysin polypeptide comprises the amino acid sequence of SEQ ID NO: 6. In certain embodiments, the nucleic acid molecule encodes a modified lysin polypeptide having at least 80% sequence identity with SEQ ID NO: 6, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85%, 85%, 90%, 95%, 98%, or 99% sequence identity with SEQ ID NO: 6.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, and N184D. In certain embodiments, the nucleic acid molecule encodes a modified lysin polypeptide, wherein the modified lysin polypeptide comprises the amino acid sequence of SEQ ID NO: 7. In certain embodiments, the nucleic acid molecule encodes a modified lysin polypeptide having at least 80%, 85%, 90%, 95%, 98%, or 99% sequence identity with SEQ ID NO: 7, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164N, and R195E. In certain embodiments, the nucleic acid molecule encodes a modified lysin polypeptide, wherein the modified lysin polypeptide comprises the amino acid sequence of SEQ ID NO: 8. In certain embodiments, the nucleic acid molecule encodes a modified lysin polypeptide having at least 80%, 85%, 90%, 95%, 98%, or 99% sequence identity with SEQ ID NO: 8, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, N184D, and S198H. In certain embodiments, the nucleic acid molecule encodes a modified lysin polypeptide, wherein the modified lysin polypeptide comprises the amino acid sequence of SEQ ID NO: 9. In certain embodiments, the nucleic acid molecule encodes a modified lysin polypeptide having at least 80%, 85%, 90%, 95%, 98%, or 99% sequence identity with SEQ ID NO: 9, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, N184D, V204A, and V212A. In certain embodiments, the nucleic acid molecule encodes a modified lysin polypeptide, wherein the modified lysin polypeptide comprises the amino acid sequence of SEQ ID NO: 10. In certain embodiments, the nucleic acid molecule encodes a modified lysin polypeptide having at least 80%, 85%, 90%, 95%, 98%, or 99% sequence identity with SEQ ID NO: 10, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: Y164N, N184D, R195E, V204K, and V212E. In certain embodiments, the nucleic acid molecule encodes a modified lysin polypeptide, wherein the modified lysin polypeptide comprises the amino acid sequence of SEQ ID NO: 11. In certain embodiments, the nucleic acid molecule encodes a modified lysin polypeptide having at least 80%, 85%, 90%, 95%, 98%, or 99% sequence identity with SEQ ID NO: 11, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: R35E, L92W, V104S, V128T, and Y137S. In certain embodiments, the nucleic acid molecule encodes a modified lysin polypeptide, wherein the modified lysin polypeptide comprises the amino acid sequence of SEQ ID NO: 12. In certain embodiments, the nucleic acid molecule encodes a modified lysin polypeptide having at least 80%, 85%, 90%, 95%, 98%, or 99% sequence identity with SEQ ID NO: 12, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: V128T, Y137S, and Y164K. In certain embodiments, the nucleic acid molecule encodes a modified lysin polypeptide, wherein the modified lysin polypeptide comprises the amino acid sequence of SEQ ID NO: 13. In certain embodiments, the nucleic acid molecule encodes a modified lysin polypeptide having at least 80%, 85%, 90%, 95%, 98%, or 99% sequence identity with SEQ ID NO: 13, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, and Y164K. In certain embodiments, the nucleic acid molecule encodes a modified lysin polypeptide, wherein the modified lysin polypeptide comprises the amino acid sequence of SEQ ID NO: 14. In certain embodiments, the nucleic acid molecule encodes a modified lysin polypeptide having at least 80%, 85%, 90%, 95%, 98%, or 99% sequence identity with SEQ ID NO: 14, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, V204K, and V212E. In certain embodiments, the nucleic acid molecule encodes a modified lysin polypeptide, wherein the modified lysin polypeptide comprises the amino acid sequence of SEQ ID NO: 15. In certain embodiments, the nucleic acid molecule encodes a modified lysin polypeptide having at least 80%, 85%, 90%, 95%, 98%, or 99% sequence identity with SEQ ID NO: 15, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, N184D, S198Q, V204K, and V212E. In certain embodiments, the nucleic acid molecule encodes a modified lysin polypeptide, wherein the modified lysin polypeptide comprises the amino acid sequence of SEQ ID NO: 16. In certain embodiments, the nucleic acid molecule encodes a modified lysin polypeptide having at least 80%, 85%, 90%, 95%, 98%, or 99% sequence identity with SEQ ID NO: 16, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, 1206E, and V214G. In certain embodiments, the nucleic acid molecule encodes a modified lysin polypeptide, wherein the modified lysin polypeptide comprises the amino acid sequence of SEQ ID NO: 17. In certain embodiments, the nucleic acid molecule encodes a modified lysin polypeptide having at least 80%, 85%, 90%, 95%, 98%, or 99% sequence identity with SEQ ID NO: 17, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1).

### Vectors and Host Cells

In another aspect, the present disclosure is directed to a vector comprising an isolated polynucleotide comprising a nucleic acid molecule encoding the lysin polypeptides disclosed herein or a complementary sequence of the present isolated polynucleotides. In some embodiments, the vector is a plasmid or cosmid. In other embodiments, the vector is a viral vector, wherein additional DNA segments can be ligated into the viral genome. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors,” can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

Generally, any system or vector suitable to maintain, propagate or express a polypeptide in a host may be used for expression of the lysin polypeptide disclosed herein or fragments thereof. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001). Additionally, tags can also be added to the lysin polypeptide of the present disclosure or fragments thereof to provide convenient methods of isolation, e.g., c-myc, biotin, poly-His, etc. Kits for such expression systems are commercially available.

A wide variety of host/expression vector combinations may be employed in expressing the polynucleotide sequences encoding the present lysin polypeptides. Large numbers of suitable vectors are known to those of skill in the art, and are commercially available. Examples of suitable vectors are provided, e.g., in Sambrook et al, eds., Molecular Cloning: A Laboratory Manual (3rd Ed.), Vols. 1-3, Cold Spring Harbor Laboratory (2001). Such vectors include, among others, chromosomal, episomal and virus derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Furthermore, the vectors may provide for the constitutive or inducible expression of the lysin polypeptide of the present disclosure. Suitable vectors include but are not limited to derivatives of SV40 and known bacterial plasmids, e.g., E. coli plasmids colE1, pCR1, pBR322, pMB9 and their derivatives, plasmids such as RP4, pBAD24 and pBAD-TOPO; phage DNAS, e.g., the numerous derivatives of phage A, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs Inc., Addgene, Takara Bio Inc., ThermoFisher Scientific Inc., etc.

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding the lysin polypeptide of the present disclosure. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof. Typically, the polynucleotide sequences encoding the lysin polypeptide or fragments thereof are operatively linked to a heterologous promoter or regulatory element.

In another aspect, the present disclosure is directed to an isolated host cell comprising any of the vectors disclosed herein including the expression vectors comprising the polynucleotide sequences encoding the lysin polypeptides of the present disclosure. A wide variety of host cells are useful in expressing the present polypeptides. Non-limiting examples of host cells suitable for expression of the present polypeptides include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

While the expression host may be any known expression host cell, in a typical embodiment the expression host is one of the strains of E. coli. These include, but are not limited to commercially available E. coli strains such as Top10 (ThermoFisher Scientific, Inc.), DH5a (Thermo Fisher Scientific, Inc.), XLI-Blue (Agilent Technologies, Inc.), SCSllO (Agilent Technologies, Inc.), JM109 (Promega, Inc.), LMG194 (ATCC), and BL21 (Thermo Fisher Scientific, Inc.). There are several advantages of using E. coli as a host system including: fast growth kinetics, where under the optimal environmental conditions, its doubling time is about 20 min (Sezonov et al., J. Bacterial. 189 8746-8749 (2007)), easily achieved high density cultures, easy and fast transformation with exogenous DNA, etc. Details regarding protein expression in E. coli, including plasmid selection as well as strain selection are discussed in details by Rosano, G. and Ceccarelli, E., Front Microbial., 5: 172 (2014).

Efficient expression of the present lysin polypeptides depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding the lysin polypeptides of the present disclosure, one skilled in the art will be able to select the proper vectors, expression control sequences, and hosts without undue experimentation to accomplish the desired expression without departing from the scope of this disclosure.

The lysin polypeptides of the present disclosure can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also employed for lysin polypeptide purification.

Alternatively, the vector system used for the production of the lysin polypeptides of the present disclosure may be a cell-free expression system. Various cell-free expression systems are commercially available, including, but are not limited to those available from Promega, LifeTechnologies, Clonetech, etc.

### Compositions Comprising Lysin Polypeptides

The lysin polypeptides disclosed herein may be incorporated into antimicrobial and bactericidal compositions and unit dosage forms thereof alone or with one or more conventional antibiotics and other bactericidal agents.

Typically, the compositions contain the lysin polypeptide as disclosed herein in an amount effective for killing Gram-positive bacteria. In certain embodiments, the Gram-positive bacteria is selected from the group consisting of Staphylococcus aureus; Listeria monocytogenes; a coagulase negative staphylococcus such as from the Staphylococcus epidermidis group, the Staphylococcus saprophyticus group, the Staphylococcus simulans group, the Staphylococcus intermedius group, the Staphylococcus sciuri group, and the Staphylococcus hyicus group; Streptococcus suis; Streptococcus pyogenes; Streptococcus agalactiae; Streptococcus dysgalactiae; Streptococcus pneumoniae; species included in the viridans streptococci group such as the Streptococcus anginosis group, Streptococcus mitis group, Streptococcus sanguinis group, Streptococcus bovis group, Streptococcus salivarius group, and Streptococcus mutans group; Enterococcus faecalis; and Enterococcus faecium.

The compositions disclosed herein can take the form of solutions, suspensions, emulsions, tablets, pills, pellets, capsules, capsules containing liquids, powders, sustained-release formulations, suppositories, tampon applications, aerosols, sprays, lozenges, troches, candies, injectables, chewing gums, ointments, smears, time-release patches, liquid-absorbed wipes, and combinations thereof. Hence, the compositions can be employed as solids, such as tablets, lyophilized powders for reconstitution, liposomes or micelles, or the compositions can be employed as liquids, such as solutions, suspensions, gargles, emulsions, or capsules filled solids or liquids, such as for oral use. In certain embodiments, the compositions can be in the form of suppositories or capsules for rectal administration or in the form of sterile injectable or inhalable solutions or suspensions for parenteral (including, for example, intravenous or subcutaneous) or topical, such as dermal, nasal, pharyngeal or pulmonary, use. Such compositions include pharmaceutical compositions, and unit dosage forms thereof may comprise conventional or new ingredients in conventional or special proportions, with or without additional active compounds or principles. Such unit dosage forms may contain any suitable effective amount of the active ingredient commensurate with the intended daily dosage range to be employed.

Carriers and excipients can be selected from a great variety of substances acceptable for human or veterinary use. Non-limiting examples of pharmaceutically acceptable carriers or excipients include any of the standard pharmaceutical carriers, such as phosphate buffered saline solutions, water, polyols, disaccharides or polysaccharides, and emulsions such as oil/water emulsions and microemulsions. Other stabilizing excipients include proprietary blends of stabilizing and protecting solutions (SPS), cyclodextrins and recombinant human albumin (rHSA). Other excipients may include bulking agents, buffering agents, tonicity modifiers (e.g., salts and amino acids), surfactants, preservatives, antioxidants, and co-solvents. For solid oral compositions comprising a lysin polypeptide disclosed herein, suitable pharmaceutically acceptable excipients include, but are not limited to, starches, sugars, diluents, granulating agents, lubricants, binders, disintegrating agents, and the like. For liquid oral compositions, suitable pharmaceutically acceptable excipients may include, but are not limited to, water, glycols, oils, alcohols, flavoring agents, preservatives, and the like. For topical solid compositions such as creams, gels, foams, ointments, or sprays, suitable excipients may include, but are not limited to a cream, a cellulosic, or an oily base, emulsifying agents, stiffening agents, rheology modifiers or thickeners, surfactants, emollients, preservatives, humectants, alkalizing or buffering agents, and solvents.

For example, the lysin polypeptide disclosed herein can be combined with buffers that maintain the pH of a liquid suspension, solution, or emulsion within a range that does not substantially affect the activity of the lysin polypeptide. For example, a desirable pH range of the composition or of the environment wherein the active ingredient is found upon administration may be between about 4.0 and about 9.0, for example between about 4.5 and about 8.5.

A stabilizing buffer may be optionally included to permit the lysin polypeptide to exert its activity in an optimized fashion. The buffer may contain a reducing reagent, such as dithiothreitol. The stabilizing buffer may also be or include a metal chelating reagent, such as ethylenediaminetetracetic acid disodium salt, or it may contain a phosphate or citrate-phosphate buffer, or any other buffering agent, such as Tris or succinate.

A mild surfactant can be included in a pharmaceutical composition in an amount effective to potentiate the therapeutic effect of the lysin polypeptides used in the composition. Suitable mild surfactants may include, inter alia, esters of polyoxyethylene sorbitan and fatty acids (such as the Tween series), octylphenoxy polyethoxy ethanol (such as the Triton-X series), n-Octyl-β-D-glucopyranoside, n-Octyl-β-D-thioglucopyranoside, n-Decyl-β-D-glucopyranoside, n-Dodecyl-β-D-glucopyranoside, poloxamer, polysorbate 20, polysorbate 80, polyethylene glycol, and biologically occurring surfactants, e.g., fatty acids, glycerides, monoglycerides, deoxycholate, and esters of deoxycholate.

Preservatives may also be used in the compositions disclosed herein, and may, for example, comprise about 0.05% to about 0.5% by weight of the total composition. The use of preservatives may assure that if the product is microbially-contaminated, the formulation will prevent or diminish microorganism growth (or attenuate the potency of the formulation). Exemplary preservatives include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

For oral administration, the lysin polypeptides disclosed herein can be formulated into solid or liquid preparations, for example tablets, capsules, powders, solutions, suspensions, and dispersions. For oral administration in the form of a tablet or capsule, the active ingredient may be combined with one or more pharmaceutically acceptable excipients such as binding agents (e.g., pregelatinized maize starch, polyvinylpyrrolidone, or hydroxypropyl methylcellulose); fillers (e.g., lactose, sucrose, glucose, mannitol, sorbitol, other reducing and non-reducing sugars, microcrystalline cellulose, calcium sulfate, or calcium hydrogen phosphate); lubricants (e.g., magnesium stearate, talc, silica, steric acid, sodium stearyl fumarate, glyceryl behenate, calcium stearate, and the like); disintegrants (e.g., potato starch or sodium starch glycolate); wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the drug components can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (e.g., BHA, BHT, propyl gallate, sodium ascorbate, or citric acid) can also be added to stabilize the dosage forms.

In certain embodiments, the tablets can be coated by methods well-known in the art. The compositions disclosed herein can be also introduced in microspheres or microcapsules, e.g., fabricated from polyglycolic acid/lactic acid (PGLA). Liquid preparations for oral administration can take the form of, for example, solutions, syrups, emulsions, or suspensions, or they can be presented as a dry product for reconstitution with water or other suitable vehicle before use. Preparations for oral administration can be suitably formulated to give controlled or postponed release of the active compound.

The active agents can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines, as is well known.

For preparing solid compositions such as tablets and pills, a lysin polypeptide as disclosed herein may be mixed with a pharmaceutical excipient to form a solid preformulation composition. If desired, tablets may be sugar coated or enteric coated by standard techniques. The tablets or pills may be coated or otherwise compounded to provide a dosage form affording the advantage of prolonged or delayed action. For example, the tablet or pill can include an inner dosage and an outer dosage component, the latter being in the form of an envelope over the former. The two components can be separated by an enteric layer, which serves to resist disintegration in the stomach and permit the inner component to pass intact into the duodenum or to be further delayed in release. A variety of materials can be used for such enteric layers or coatings, such materials including a number of polymeric acids and mixtures of polymeric acids with such materials as shellac, cetyl alcohol, and cellulose acetate. Similarly, the orally-administered medicaments may be administered in the form of a time-controlled release vehicle, including diffusion-controlled systems, osmotic devices, dissolution-controlled matrices, and erodible/degradable matrices.

Topical compositions as disclosed herein may further comprise a pharmaceutically or physiologically acceptable carrier, such as a dermatologically or an otically acceptable carrier. Such carriers, in the case of dermatologically acceptable carriers, may be compatible with skin, nails, mucous membranes, tissues, and/or hair, and can include any conventionally used dermatological carrier meeting these requirements. In the case of otically acceptable carriers, the carrier may be compatible with all parts of the ear. Such carriers can be readily selected by one of ordinary skill in the art. Carriers for topical administration of the compounds disclosed herein include, but are not limited to, mineral oil, liquid petroleum, white petroleum, propylene glycol, polyoxyethylene and/or polyoxypropylene compounds, emulsifying wax, sorbitan monostearate, polysorbate 60, cetyl esters wax, cetearyl alcohol, 2-octyldodecanol, benzyl alcohol, and water. In formulating skin ointments, the active components of the present disclosure may be formulated in an oleaginous hydrocarbon base, an anhydrous absorption base, a water-in-oil absorption base, an oil-in-water water-removable base, and/or a water-soluble base. In formulating otic compositions, the active components of the present disclosure may be formulated in an aqueous polymeric suspension including such carriers as dextrans, polyethylene glycols, polyvinylpyrrolidone, polysaccharide gels, Gelrite®, cellulosic polymers like hydroxypropyl methylcellulose, and carboxy-containing polymers such as polymers or copolymers of acrylic acid, as well as other polymeric demulcents. The topical compositions as disclosed herein may be in any form suitable for topical application, including aqueous, aqueous-alcoholic or oily solutions; lotion or serum dispersions; aqueous, anhydrous or oily gels; emulsions obtained by dispersion of a fatty phase in an aqueous phase (O/W or oil in water) or, conversely, dispersion of an aqueous phase in a fatty phase (W/O or water in oil), microemulsions or alternatively microcapsules, microparticles or lipid vesicle dispersions of ionic and/or nonionic type, creams, lotions, gels, foams (which may use a pressurized canister, a suitable applicator, an emulsifier, and an inert propellant), essences, milks, suspensions, or patches. Topical compositions disclosed herein may also contain adjuvants such as hydrophilic or lipophilic gelling agents, hydrophilic or lipophilic active agents, preserving agents, antioxidants, solvents, fragrances, fillers, sunscreens, odor-absorbers, and dyestuffs. In a further aspect, the topical compositions disclosed herein may be administered in conjunction with devices such as transdermal patches, dressings, pads, wraps, matrices and bandages capable of being adhered or otherwise associated with the skin or other tissue or organ of a subject, being capable of delivering a therapeutically-effective amount of one or more lysin polypeptides or fragments thereof as disclosed herein.

In some embodiments, the topical compositions disclosed herein additionally comprise one or more components used to treat topical burns. Such components may include, but are not limited to, a propylene glycol hydrogel; a combination of a glycol, a cellulose derivative and a water-soluble aluminum salt; an antiseptic; an antibiotic; and a corticosteroid. Humectants (such as solid or liquid wax esters), absorption promoters (such as hydrophilic clays, or starches), viscosity building agents, and skin-protecting agents may also be added. Topical formulations may be in the form of rinses such as mouthwash. See, e.g., WO2004/004650.

The lysin polypeptides disclosed herein may also be administered by injection of a therapeutic agent comprising the appropriate amount of a lysin polypeptide and a carrier. For example, the lysin polypeptides can be administered intramuscularly, intracerebrovetricularly, intrathecally, subdermally, subcutaneously, intreaperitoneally, intravenously, or by direct injection or continuous infusion to treat infections by bacteria, such as Gram-positive bacteria. The carrier may be comprised of distilled water, a saline solution, albumin, a serum, or any combinations thereof. Additionally, pharmaceutical compositions of parenteral injections can comprise pharmaceutically-acceptable aqueous or nonaqueous solutions of lysin polypeptides in addition to one or more of the following: pH buffered solutions, adjuvants (e.g., preservatives, wetting agents, emulsifying agents, stabilizing agents, and dispersing agents), liposomal formulations, nanoparticles, dispersions, suspensions, and emulsions, as well as sterile powders for reconstitution into sterile injectable solutions or dispersions just prior to use.

In certain embodiments, formulations for injection can be presented in unit dosage form, e.g., in ampoules or in multi-dose containers, and in certain embodiments may include an added preservative. The compositions can take such forms as excipients, suspensions, solutions, or emulsions in oily or aqueous vehicles, and can contain formulatory agents such as suspending, stabilizing, bulking, and/or dispersing agents. The active ingredient can be in powder form for reconstitution with a suitable vehicle, e.g., sterile pyrogen-free water, before use. Examples of buffering agents may include histidine, Tris, phosphate, succinate citrate, methionine, cystine, glycine, mild surfactants, calcium, and magnesium. A reducing agent such as dithiothreitol can also be included.

In cases where parenteral injection is the chosen mode of administration, an isotonic formulation may be used. Generally, additives for isotonicity can include sodium chloride, dextrose, sucrose, glucose, trehalose, mannitol, sorbitol, and lactose. In some cases, isotonic solutions such as phosphate buffered saline may be used. Stabilizers can include histidine, methionine, glycine, arginine, gelatin, and albumin, such as human or bovine serum albumin. A person of ordinary skill will readily appreciate that many of the foregoing excipients can also be used in compositions for injection.

A vasoconstriction agent can be added to the compositions disclosed herein. In certain embodiments, the compositions may be provided sterile and pyrogen-free.

In another embodiment, the compositions disclosed herein may be dry inhalable powders or other inhalable compositions, such as aerosols or sprays. The inhalable compositions disclosed herein can further comprise a pharmaceutically acceptable carrier. For administration by inhalation, the lysin polypeptides may be conveniently delivered in the form of an aerosol spray presentation from such devices as inhalers, pressurized aerosol dispensers, or nebulizers, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the active ingredient and a suitable powder base such as lactose or starch.

In one embodiment, lysin polypeptide disclosed herein may be formulated as a dry, inhalable powder or as an aerosol or spray. In specific embodiments, a lysin polypeptide inhalation solution may further be formulated with a propellant for aerosol delivery. In certain embodiments, solutions may be nebulized. Many dispensing devices are available in the art for delivery of pharmaceutical compositions, including polypeptides, by inhalation. These include nebulizers, pressurized aerosol dispensers, and inhalers.

A surfactant can be added to an inhalable pharmaceutical composition as disclosed herein in order to lower the surface and interfacial tension between the medicaments and the propellant. Where the medicaments, propellant, and excipient are to form a suspension, a surfactant may or may not be required. Where the medicaments, propellant, and excipient are to form a solution, a surfactant may or may not be necessary, depending in part on the solubility of the particular medicament and excipient. The surfactant may be any suitable, non-toxic compound that is non-reactive with the medicament and that reduces the surface tension between the medicament, the excipient, and the propellant and/or acts as a valve lubricant.

Examples of suitable surfactants include, but are not limited to: oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl ether; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.

Examples of suitable propellants include, but are not limited to: dichlorodifluoromethane, trichlorofluoromethane, dichloro-tetrafluoroethane, and carbon dioxide.

Examples of suitable excipients for use in inhalable compositions include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

In some embodiments, the compositions disclosed herein comprise nasal applications. Nasal applications include, for instance, nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application.

Compositions disclosed herein can also be formulated for rectal administration, e.g., as suppositories or retention enemas (e.g., containing conventional suppository bases such as cocoa butter or other glycerides).

In certain embodiments, the compositions disclosed herein may further comprise at least one antibiotic, such as at least one antibiotic effective to inhibit the growth, reduce the population, or kill at least one species of Gram-positive bacteria. In certain embodiments, the at least one antibiotic is effective against one or more of Staphylococcus aureus; Listeria monocytogenes; a coagulase negative staphylococcus such as from the Staphylococcus epidermidis group, the Staphylococcus saprophyticus group, the Staphylococcus simulans group, the Staphylococcus intermedius group, the Staphylococcus sciuri group, and the Staphylococcus hyicus group; Streptococcus suis; Streptococcus pyogenes; Streptococcus agalactiae; Streptococcus dysgalactiae; Streptococcus pneumoniae; species included in the viridans streptococci group such as the Streptococcus anginosis group, Streptococcus mitis group, Streptococcus sanguinis group, Streptococcus bovis group, Streptococcus salivarius group, and Streptococcus mutans group; Enterococcus faecalis; and Enterococcus faecium.

In certain embodiments of the compositions disclosed herein, the lysin polypeptide in combination with the at least one antibiotic may exhibit synergism, for example synergism in the lysin polypeptide's, the fragment's, or the antibiotic's ability to inhibit the growth, reduce the population, or kill at least one species of Gram-positive bacteria. Synergy may refer to the inhibitory activity of a combination of two active agents, wherein the fractional inhibitory concentration (FIC) index for the combination is less than 1, and for strong synergy, less than or equal to 0.5. The FIC of an agent is the minimum concentration of that agent that kills bacteria when used in combination with another agent divided by the concentration of the first agent that has the same effect when the first agent is used alone. The FIC index for the combination of A and B is the sum of their individual FIC values.

Synergy may be evaluated in a checkerboard assay (and can be validated by time-kill curves). Each checkerboard assay generates many different combinations, and, by convention, the FIC values of the most effective combination are used in calculating the FIC index. The FIC index defines the nature of the interaction. Antimicrobial agents with additive interactions have a FIC index of 1; an FIC index of <1 defines synergistic interactions; combinations with an FIC index >1 are antagonistic. The lower the FIC index, the more synergistic a combination. See, e.g., Singh, P. K. et al, Am J Physiol Lung Cell Mol Physiol 279: L799-L805, 2000. Synergy has implications for an efficacious, new general anti-infective strategy based on the co-administration of lysin polypeptides and antibiotics. In particular each and both lysin polypeptides and antibiotics may be administered at reduced doses and amounts, with enhanced bactericidal and bacteriostatic activity and with reduced risk of resistance development. In other words, the benefits of synergy are not only realized when one or both agents are used at sub-MIC concentrations, although the existence of synergy may be revealed by testing with sub-MIC concentrations of each agent.

### Methods

The lysin polypeptides disclosed herein may be administered to a subject in need thereof, e.g., a living animal (including a human) for the treatment, alleviation, or amelioration, palliation, or elimination of an indication or condition which is susceptible thereto. In particular, as disclosed herein, the lysin polypeptides can be co-administered with at least one β-lactam antibiotic and used in a method of resensitizing a Gram-positive bacterium to the at least one (3-lactam antibiotic.

Accordingly, the lysin polypeptides of the present disclosure can be co-administered with at least one β-lactam antibiotic in vivo, for example, to treat bacterial infections due to Gram-positive bacteria, such as S. aureus, in a subject, as well as in vitro, for example to reduce the level of bacterial contamination on, for example, a surface, e.g., of a medical device and to resensitize the Gram-positive bacterium to the at least one β-lactam antibiotic.

As discussed above, antibiotic resistance may occur when bacteria that previously were sensitive to a particular antibiotic develops resistance to that antibiotic, and further administration of the antibiotic does not prevent, control, disrupt, or treat the bacterial infection. Resensitization is the ability of a bacteria to regain susceptibility to an antibiotic that the bacteria was previously resistant towards. Therefore, according to certain aspects, there is disclosed herein a method of resensitizing a Gram-positive bacterium in a subject to at least one antibiotic, such as at least one β-lactam antibiotic, the method comprising co-administering to the subject at least one antibiotic and a lysin polypeptide, thereby resensitizing the Gram-positive bacterium to the at least one antibiotic. In certain embodiments, the lysin polypeptide may be PlySs2 (SEQ ID NO: 1) or an active fragment thereof. In certain embodiments, the lysin polypeptide may be a modified lysin polypeptide having an amino acid sequence selected from the group consisting of SEQ ID NOs. 3-17.

In one aspect, the lysin polypeptide and the at least one antibiotic are administered sequentially; for example, in certain embodiments, the lysin polypeptide is administered prior to administration of the at least one antibiotic. In one aspect, the lysin polypeptide and the at least one antibiotic are administered substantially simultaneously. In certain embodiments, the at least one antibiotic is not effective to reduce the population, kill, inhibit the growth, and/or eradicate the Gram-positive bacterium prior to administration of the lysin polypeptide.

In some embodiments, the present lysin polypeptides may be co-administered with at least one β-lactam antibiotic for use in resensitizing Gram-positive bacteria that forms a biofilm to the at least one β-lactam antibiotic and the prevention, control, disruption, and treatment of bacterial biofilm formed by Gram-positive bacteria. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. Biofilm may develop in any supporting environment including living and nonliving surfaces such as the mucus plugs of the CF lung, contaminated catheters, implants, contact lenses, etc (Sharma et al. Biologicals, 42(1):1-7 (2014), which is herein incorporated by reference in its entirety). Because biofilms protect the bacteria, they are often more resistant to traditional antimicrobial treatments, making them a serious health risk, which is evidenced by more than one million cases of catheter-associated urinary tract infections (CAUTI) reported each year, many of which can be attributed to biofilm-associated bacteria (Donlan, R M (2001) Emerg Infect Dis7(2):277-281; Maki D and Tambyah P (2001) Emerg Infect Dis 7(2):342-347).

Thus, in one embodiment, the lysin polypeptides of the present disclosure can be co-administered with at least one β-lactam antibiotic and used for resensitization of the Gram-positive bacterium to the at least one β-lactam antibiotic and the prevention, control, disruption, and treatment of bacterial infections due to Gram-positive bacteria when the Gram-positive bacteria are protected by a bacterial biofilm.

In one aspect, the present disclosure is directed to a method of resensitizing a Gram-positive bacterium, as described herein, to at least one β-lactam antibiotic, comprising administering to a subject diagnosed with, at risk for, or exhibiting symptoms of a bacterial infection, a pharmaceutical composition as described herein.

The synergy data disclosed herein indicate that, in some embodiments, the present lysins will be able to drive the resensitization of Gram-positive bacteria including MDR organisms, such as MRSA as described in the Examples. Generally resensitization occurs in synergistic combinations in which the antibiotic MIC values fall below established breakpoints, e.g., a MIC value of ≤2 for antibiotic sensitive bacteria, a MIC value of 4 for intermediately sensitive bacteria and a MIC value of ≥8 for antibiotic-resistant bacteria, e.g. β-lactam-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, Pa. As used herein, a breakpoint value is a chosen concentration (e.g., mg/L) of an antibiotic that defined whether a bacterial strain is susceptible or resistant to the antibiotic. If the MIC value of the antibiotic is less than or equal to the breakpoint value, the bacteria is considered susceptible to that antibiotic.

The terms “infection” and “bacterial infection” are meant to include respiratory tract infections (RTIs), such as respiratory tract infections in patients having cystic fibrosis (CF), lower respiratory tract infections, such as acute exacerbation of chronic bronchitis (ACEB), acute sinusitis, community-acquired pneumonia (CAP), hospital-acquired pneumonia (HAP) and nosocomial respiratory tract infections; sexually transmitted diseases, such as gonococcal cervicitis and gonococcal urethritis; urinary tract infections; acute otitis media; sepsis including neonatal septisemia and catheter-related sepsis; and osteomyelitis. Infections caused by drug-resistant bacteria and multidrug-resistant bacteria are also contemplated.

Non-limiting examples of infections caused by Gram-positive bacterial may include: A) Nosocomial infections: 1. Respiratory tract infections especially in cystic fibrosis patients and mechanically-ventilated patients; 2. Bacteraemia and sepsis; 3. Wound infections, particularly those of burn victims; 4. Urinary tract infections; 5. Post-surgery infections on invasive devises; 6. Endocarditis by intravenous administration of contaminated drug solutions; 7. Infections in patients with acquired immunodeficiency syndrome, cancer chemotherapy, steroid therapy, hematological malignancies, organ transplantation, renal replacement therapy, and other conditions with severe neutropenia. B) Community-acquired infections: 1. Community-acquired respiratory tract infections; 2. Meningitis; 3. Folliculitis and infections of the ear canal caused by contaminated water; 4. Malignant otitis externa in the elderly and diabetics; 5. Osteomyelitis of the caleaneus in children; 6. Eye infections commonly associated with contaminated contact lens; 7. Skin infections such as nail infections in people whose hands are frequently exposed to water; 8. Gastrointestinal tract infections; and 9. Muscoskeletal system infections.

The one or more species of Gram-positive bacteria of the present methods may include any of the species of Gram-positive bacteria as described herein or known in the art. Typically, the species of Gram-positive bacteria may include Listeria monocytogenes, Staphylococcus aureus, coagulase negative staphylococci (including at least 40 recognized species including, but not limited to, the Staphylococcus epidermidis group, the Staphylococcus saprophyticus group, the Staphylococcus simulans group, the Staphylococcus intermedius group, the Staphylococcus sciuri group, the Staphylococcus hyicus group, and any isolates referred to as from the “unspecified species group”), Streptococcus suis, Streptococcus pyogenes, Streptococcus agalactiae, Streptococcus dysgalactiae, Streptococcus pneumoniae, any additional species included in the viridans streptococci group (including, but not limited to, all species and strains included in the Streptococcus anginosis group, Streptococcus mitis group, Streptococcus sanguinis group, Streptococcus bovis (now gallolyticus) group, Streptococcus salivarius group, and Streptococcus mutans group), Enterococcus faecalis, and Enterococcus faecium. Other examples of Gram-positive bacteria include but are not limited to the genera Actinomyces, Bacillus, Lactococcus, Mycobacterium, Corynebacterium, and Clostridium.

The lysin polypeptides or fragments thereof of the present disclosure are co-administered with one or more β-lactam antibiotics, including, but not limited to penicillin and derivatives thereof, cephalosporins, monobactams, carbapenems, and carbacephems. In certain embodiments, the at least one β-lactam antibiotic may be chosen from penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, piperacillin, cefazolin, cephalexin, cephalosporin, cephalothin, cefaclor, cefamandole, cefuroxime, cefotetan, cefoxitin, cefixime, cefotaxime, cefpodoxime, ceftazidime, ceftriaxone, cefdinir, cefepime, cefpirome, ceftaroline, biapenem, doripenem, ertapenem, faropenem, imipenem, meropenem, panipenem, razupenem, tebipenem, and thienamycin. In certain embodiments, the at least one β-lactam antibiotic may be chosen from oxacillin, nafcillin, cefazolin, and methicillin. In certain embodiments, it may be desirable to administer or more additional standard care antibiotics or with antibiotics of last resort, individually or in various combinations as within the skill of the art. Traditional antibiotics used against Gram-positive bacteria, other than β-lactam antibiotics, are described herein and may include, for example, vancomycin, daptomycin, mupirocin, lysostaphin, penicillins, cloxacillin, erythromycin, carbapenems, cephalosporins, glycopeptides, lincosamides, azithromycin, clarithromycin, roxithromycin, telithromycin, spiramycin, and fidaxomicin.

Combining the lysin polypeptide of the present disclosure with at least one β-lactam antibiotic provides an efficacious antibacterial regimen. In some embodiments, co-administration of the lysin polypeptide or active fragment thereof of the present disclosure with one or more β-lactam antibiotics may be carried out at reduced doses and amounts of either the lysin polypeptide or the β-lactam antibiotic or both, and/or reduced frequency and/or duration of treatment with augmented bactericidal and bacteriostatic activity, reduced risk of antibiotic resistance and with reduced risk of deleterious neurological or renal side effects. As used herein the term “reduced dose” refers to the dose of one active ingredient in the combination compared to monotherapy with the same active ingredient. In some embodiments, the dose of the lysin polypeptide or the β-lactam antibiotic in a combination may be suboptimal or even subthreshold compared to the respective monotherapy.

In some embodiments, the present disclosure provides a method of augmenting antibiotic activity of one or more β-lactam antibiotics against Gram-positive bacteria compared to the activity of said β-lactam antibiotics used alone by administering to a subject one or more lysin polypeptide disclosed herein together with a β-lactam antibiotic of interest. Co-administering the lysin polypeptide and β-lactam antibiotic is effective against the Gram-positive bacteria and permits resistance against the antibiotic to be overcome and/or the antibiotic to be employed at lower doses, decreasing undesirable side effects.

In some embodiments of the method of resensitizing a Gram-positive bacterium to the at least one β-lactam antibiotic, the method comprises contacting Gram-positive bacteria with the lysin polypeptide and at least one β-lactam antibiotic as described herein, wherein the Gram-positive bacteria are present on a surface of e.g., medical devices, floors, stairs, walls and countertops in hospitals and other health related or public use buildings and surfaces of equipment in operating rooms, emergency rooms, hospital rooms, clinics, and bathrooms and the like.

Examples of medical devices that can be protected using the methods described herein include but are not limited to tubing and other surfaces of medical devices, such as urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-positive bacteria.

### Dosages and Administration

Dosages administered depend on a number of factors such as the activity of infection being treated; the age, health and general physical condition of the subject to be treated; the activity of a particular lysin polypeptide; the nature and activity of the antibiotic if any with which a lysin polypeptide according to the present disclosure is being paired; and the combined effect of such pairing. In certain embodiments, effective amounts of the lysin polypeptide or fragment thereof to be administered may fall within the range of about 0.1-100 mg/kg (or 1 to 100 mcg/ml), such as from 0.5 mg/kg to 30 mg/kg. In certain embodiments, the lysin polypeptide may be administered 1-4 times daily for a period ranging from 1 to 14 days. The antibiotic may be administered at standard dosing regimens or in lower amounts in view of any synergism. All such dosages and regimens, however, (whether of the lysin polypeptide or any antibiotic administered in conjunction therewith) are subject to optimization. Optimal dosages can be determined by performing in vitro and in vivo pilot efficacy experiments as is within the skill of the art but taking the present disclosure into account.

It is contemplated that the lysin polypeptide disclosed herein may provide a rapid bactericidal and, when used in sub-MIC amounts, may provide a bacteriostatic effect. It is further contemplated that the lysin polypeptide disclosed herein may be active against a range of antibiotic-resistant bacteria. Based on the present disclosure, in a clinical setting, the present lysin polypeptide may be a potent additive for treating infections arising from drug- and multidrug-resistant bacteria and overcoming resistance to β-lactam antibiotics.

In some embodiments, time exposure to the lysin polypeptide disclosed herein may influence the desired concentration of active polypeptide units per ml. Carriers that are classified as “long” or “slow” release carriers (such as, for example, certain nasal sprays or lozenges) may possess or provide a lower concentration of polypeptide units per ml but over a longer period of time, whereas a “short” or “fast” release carrier (such as, for example, a gargle) may possess or provide a high concentration of polypeptide units (mcg) per ml but over a shorter period of time. There are circumstances where it may be desirable to have a higher unit/ml dosage or a lower unit/ml dosage.

For the lysin polypeptide of the present disclosure and the β-lactam antibiotic, the therapeutically effective dose may be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration, in humans. Dosage and administration can be further adjusted to provide sufficient levels of the active ingredient or to maintain the desired effect. Additional factors that may be taken into account include the severity of the disease state; age, weight and gender of the patient; diet; desired duration of treatment; method of administration; time and frequency of administration; drug combinations; reaction sensitivities; tolerance/response to therapy; and the judgment of a treating physician.

A treatment regimen can entail daily administration (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration may be used. Dosage and frequency may vary depending on the patient. It will be understood by one of skill in the art that such guidelines will be adjusted for localized administration, e.g., intranasal, inhalation, rectal, etc., or for systemic administration, e.g., oral, rectal (e.g., via enema), intramuscular (i.m.), intraperitoneal (i.p.), intravenous (i.v.), subcutaneous (s.c.), transurethral, and the like.

Specific embodiments disclosed herein may be further limited in the claims using “consisting of” and/or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein. The applicants reserve the right to disclaim any embodiment or feature described herein.

### EXAMPLES

The methods and lysin polypeptides described herein and their preparation, characterization, and use will be better understood in connection with the following examples, which are intended as an illustration of and not a limitation upon the scope of the present disclosure.

### Example 1—Synergy Between PlySs2 Lysin and β-Lactam Antibiotics

A step-wise approach was used to evaluate PlySs2 as a resensitizing agent. First, broth microdilution checkerboard assays were used to determine fractional inhibitory concentration index (FICI) values for combinations of PlySs2 with three β-lactam antibiotics [oxacillin (OXA), nafcillin (NAF), and cefazoline (CFZ)] against nine different MRSA strains. Data from the checkerboard assays were generated to determine the interaction and potency of PlySs2 with the β-lactam antibiotics in comparison to their individual activities. This comparison is represented as the FICI value, whereby values of ≤0.5 are consistent with synergy, values of >0.5-<1 are highly-additive, values of 1-≤2 are indifferent, and values >2 are antagonistic. Representative single agent MICs are also shown, determined for each agent alone (initial) and in combinations (final). Resensitization occurs in synergistic combinations in which the β-lactam antibiotic MIC values fall below established breakpoints, e.g. a MIC value of ≤2 for β-lactam-sensitive isolates, a MIC value of ≥4 for β-lactam-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, Pa.

As indicated in Table 1 below, synergistic combinations with PlySs2 demonstrated reductions of OXA, NAF, and CFZ MICs to below breakpoint values for each of the nine MRSA strains examined. These observations are consistent with resensitization. The ability of PlySs2 lysins to resensitize antibiotic-resistant bacterial strains to conventional antibiotics indicates the benefit of these biologics as therapeutics to combat and reverse antimicrobial resistance.

TABLE 1
Antibactericidal Activity of PlySs2 and β-lactam antibiotics,
alone and in combination, against MRSA strains

| MRSA | | Antimicrobial agents | | | | | |
|---|---|---|---|---|---|---|---|
| Strain | MIC/FICI | PlySs2 | OXA | PlySs2 | NAF | PlySs2 | CFZ |
| NRS 11 | MICᵢₙᵢₜᵢₐₗ | 1 | 256 | 1 | 64 | 1 | 256 |
| | MIC_{final} | 0.25 | 2* | 0.25 | 1* | 0.25 | 1* |
| | FICI | 0.258† | | 0.266† | | 0.254† | |
| ATCC | MICᵢₙᵢₜᵢₐₗ | 1 | 8 | 1 | 2 | 1 | 16 |
| 43300 | MIC_{final} | 0.25 | 0.5* | 0.125 | 0.25 | 0.125 | 4 |
| | FICI | 0.313† | | 0.250† | | 0.133† | |
| HPV | MICᵢₙᵢₜᵢₐₗ | 1 | 8 | 1 | 2 | 1 | 16 |
| 107 | MIC_{final} | 0.25 | 0.5* | 0.25 | 0.25 | 0.125 | 2* |
| | FICI | 0.313† | | 0.375† | | 0.250† | |
| CAIRD | MICᵢₙᵢₜᵢₐₗ | 1 | 4 | 1 | 16 | 1 | 8 |
| 426 | MIC_{final} | 0.25 | 1* | 0.25 | 1* | 0.25 | 0.5* |
| | FICI | 0.313† | | 0.313† | | 0.313† | |
| JMI 227 | MICᵢₙᵢₜᵢₐₗ | 1 | 16 | 1 | 4 | 1 | 2 |
| | MIC_{final} | 0.25 | 1* | 0.25 | 0.5* | 0.25 | 0.5 |
| | FICI | 0.313† | | 0.375† | | 0.500† | |
| JMI | MICᵢₙᵢₜᵢₐₗ | 1 | 256 | 1 | 256 | 1 | 32 |
| 1280 | MIC_{final} | 0.25 | 1* | 0.25 | 2* | 0.25 | 0.5* |
| | FICI | 0.313† | | 0.258† | | 0.266† | |
| JMI4789 | MICᵢₙᵢₜᵢₐₗ | 1 | 64 | 1 | 4 | 1 | 4 |
| | MIC_{final} | 0.25 | 2* | 0.125 | 0.5* | 0.125 | 1* |
| | FICI | 0.281† | | 0.250† | | 0.375† | |
| MW2 | MICᵢₙᵢₜᵢₐₗ | 1 | 64 | 2 | 4 | 1 | 4 |
| | MIC_{final} | 0.25 | 2* | 0.5 | 0.031* | 0.125 | 1* |
| | FICI | 0.281† | | 0.258† | | 0.375† | |
| ATCC | MICᵢₙᵢₜᵢₐₗ | 1 | 256 | 1 | 64 | 1 | 128 |
| 33591 | MIC_{final} | 0.25 | 1* | 1.125 | 2* | 0.25 | 0.5* |

| | | | |
|---|---|---|---|
| FICI | 0.256† | 0.156† | 0.254† |

*Resensitization
†Synergy

As shown in Table 1, all combinations of PlySs2 and each β-lactam antibiotic exhibited synergy against the 9 MRSA strains evaluated. Moreover, β-lactam sensitivity was restored to the MRSA strains, as demonstrated by the reduction of MIC values to below established β-lactam breakpoints for S. aureus.

### Example 2—In Vitro PlySs2 Lysin Exposure Increases Oxacillin Susceptibility

Serial passage resistance studies were undertaken to assess the ability of PlySs2 lysin to suppress the emergence of antibiotic resistance when used in combination with oxacillin used to treat S. aureus infections. Methods used to perform serial passage experiments are described in Palmer et al., Genetic basis for daptomycin resistance in enterococci, ANTIMICROBIAL AGENTS AND CHEMOTHERAPY (2011); 55:3345-56 and Berti et al., Altering the Proclivity towards Daptomycin Resistance in Methicillin-Resistant Staphylococcus aureus Using Combinations with Other Antibiotics, ANTIMICROBIAL AGENTS AND CHEMOTHERAPY (2012); 56:5046-53, respectively. Increases in the MIC values were assessed for a MRSA S. aureus strain (MW2) grown either in the presence of oxacillin or in the presence of a 1.1-fold dilution or a 2-fold dilution of PlySs2 lysin.

21-day in vitro serial passage resistance assays were performed to determine the impact of PlySs2 (alone) on oxacillin and PlySs2 MIC values and the potential for a “seesaw” effect similar to that previously shown, whereby exposures to daptomycin or vancomycin were accompanied by increased susceptibility (and the potential for resensitization) to β-lactam antibiotics [Renzoni et al., Molecular Bases Determining Daptomycin Resistance-Mediated Resensitizatoin to β-Lactams (Seesaw Effect) in Methicillin-Resistant Staphylococcus aureus, ANTIMICROBIAL AGENTS AND CHEMOTHERAPY (2017) 61(1):e01634-16 and Werth et al., Evaluation of Ceftaroline Activity against Heteroresistant Vancomycin-Intermediate Staphylococcus aureus and Vancomycin-Intermediate Methicillin-Resistant S. aureus Strains in an In Vitro Pharmacokinetic/Pharmacodynamic Model: Exploring the ‘Seesaw Effect’, ANTIMICROBIAL AGENTS AND CHEMOTHERAPY (2013); 57(6):2664-68].

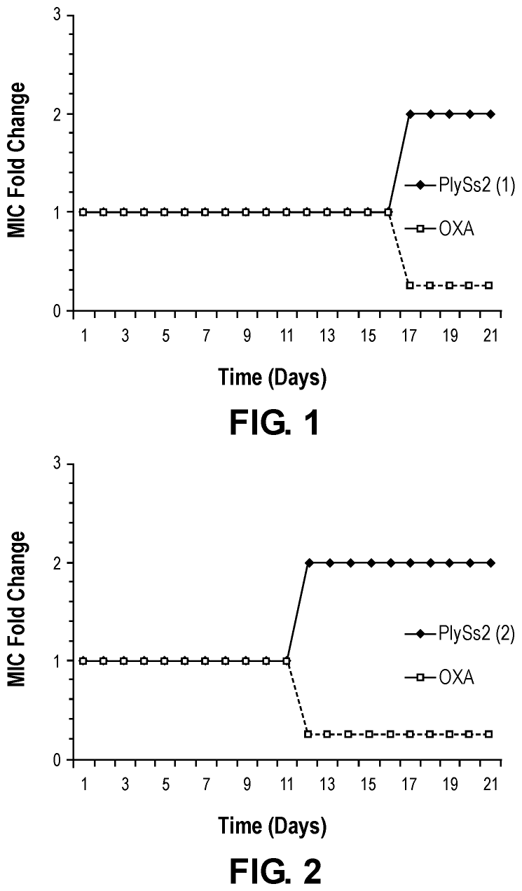

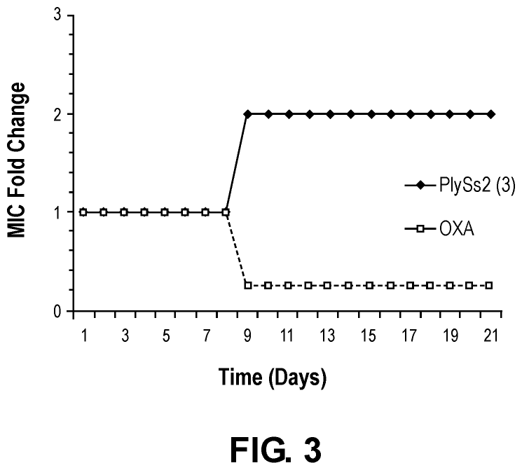

MRSA strain MW2 was serially passaged in triplicate on a daily basis for 21 days using both a 1.1-fold and 2-fold PlySs2 dilution series. As shown in FIGS. 1-3, only modest 2-fold shifts in PlySs2 MIC values were observed. PlySs2 exposure resulted in a seesaw effect, with reduced OXA MICs (0.25 MIC fold change from 64 μg/mL to 16 μg/mL). See FIGS. 1-3. This seesaw effect, i.e., a decrease in MRSA's susceptibility to PlySs2 accompanied by a paradoxical increase in susceptibility to oxacillin, indicates PlySs2 lysin's ability to resensitize MRSA to oxacillin. Three MRSA MW2 strain isolates were taken just prior (days 16, 11, and 8 for FIGS. 1-3, respectively) and just after (days 17, 12, and 9 for FIGS. 1-3, respectively) the observed MIC shift for whole genome sequencing.

It is known that the ability of daptomycin to resensitize MRSA to oxacillin is driven by mprF-mediated cell membrane modifications that result in mislocalization of factors required for maturation of PBP 2a (mecA product) [Renzono et al. (2017)]. To initiate similar studies of the PlySs2 effect, three mutant derivatives obtained just after the shift in PlySs2 and OXA MIC values (see days 17, 12, and 9 for FIGS. 1-3, respectively) were analyzed by whole genome sequencing (WGS) and SNP/INDELs; likewise, control strains obtained just prior to the shift in MIC values (days 16, 11, and 8 for FIGS. 1-3, respectively) were analyzed and compared to the mutant strains. Three distinct mutations were implicated, as shown below in Table 2, and the impact of each mutation on PlySs2 and OXA MICs was confirmed using a two-step allelic exchange process in a clean genetic background, as described in Abdelhamed et al, A novel suicide plasmid for efficient gene mutation in Listeria monocytogenes, PLASMID (2015); 81:1-8.

TABLE 2
Mutations associated with PlySs2-mediated reductions in OXA MICs

| | | | | Amino | | | | |
|---|---|---|---|---|---|---|---|---|
| Ref. | Overlapping | | | Acid | PlySs2 | PlySs2 | PlySs2 | PlySs2 |
| Position^{a} | Annotation^{b} | Ref. | Allele | Change | Control | (1) | (2) | (3) |
| 2180631 | murA | G | T | R95S | − | − | + | − |
| 2403752 | lyrA | C | A | Y245* | − | − | − | + |
| 2658191 | oatA | C | A | oatA | − | + | − | − |
| | | | | promoter | | | | |

^{a}Position in the reference genome of S. aureus MW2 (GenBank accession: NC_003923.1)
^{b}Annotated open reading frames overlapping computationally-predicted polymorphisms

As shown in Table 2, mutations in or near loci encoding three different cell wall modifying enzymes (i.e., murA, lyrA, and oatA) were each independently sufficient to reduce oxacillin MICs. These findings are consistent with a model in which cell wall perturbations, which are mediated through murA, lyrA, and/or oatA for PlySs2, reduce membrane amounts of penicillin-binding protein 2a (PBP 2a), as was observed for mprF and daptomycin [Renzoni et al. (2017)]. Although not wishing to be bound by theory, it is likewise hypothesized that exposure to PlySs2 may mediate a reduction in PBP 2a.

### Example 3—Ex Vivo PlySs2 Exposures Enhance the Increase in Oxacillin Susceptibility

An ex vivo analysis was performed on tissue samples recovered after PlySs2 treatment in a standard rabbit model of MRSA infective endocarditis (IE), as disclosed in Xiong et al., Comparative efficacy of telavancin and daptomycin in experimental endocarditis due to multi-clonotype MRSA strains, J. ANTIMIC. CHEMO. (2016); 71(1):2890-94. The standard rabbit IE model was used to confirm the impact of PlySs2 treatment on oxacillin MICs. Four days after treatment with a single-dose of PlySs2 (0.18 mg/kg to 1.4 mg/kg) in the IE model, isolates were recovered from valvular vegetations and plated on TSAB (non-selective condition, Tables 3 and 4) and TSAB supplemented with PlySs2 over a range of concentrations (selective conditions, Tables 5 and 6). MIC values of the MRSA isolates were determined for both PlySs2 and oxacillin. Tables 3 and 4 below show the MICs calculated for PlySs2 and oxacillin, respectively, for valvular vegetations subject to the non-selective conditions. It is noted that the PlySs2 MIC of S. aureus strain MW2 is 1 μg/mL, and the oxacillin MIC of S. aureus strain MW2 is 32 μg/mL.

TABLE 3
PlySs2 MICs

| | Log₁₀ | | | | | |
|---|---|---|---|---|---|---|
| | CFU/g of | PlySs2 MIC (μg/mL) | | | | |
| Treatment Group | Vegetation | 0.25 | 0.5 | 1 | 2 | 4 |
| Pre-treatment control (n = 24) | 7.02 ± 1.47 | | | 24 | | |
| Buffer treatment control (n = 32) | 7.26 ± 1.54 | | | 31 | 1 | |
| PlySs2 at 1.4 mg/kg (n = 24) | 8.24 ± 0.02 | | | 24 | | |
| PlySs2 at 0.7 mg/kg (n = 24) | 7.93 ± 0.12 | | 1 | 23 | | |
| PlySs2 at 0.35 mg/kg (n = 24) | 8.17 ± 0.58 | | 3 | 21 | | |
| PlySs2 at 0.18 mg/kg (n = 24) | 8.65 ± 0.58 | | | 16 | | |

TABLE 4
Oxacillin MICs

| | Log₁₀ | | | | | | |
|---|---|---|---|---|---|---|---|
| | CFU/g of | Oxacillin MIC (μg/mL) | | | | | |
| Treatment Group | Vegetation | ≤2 | 4 | 8 | 16 | 32 | 64 |
| Pre-treatment control (n = 24) | 7.02 ± 1.47 | | | | | 24 | |
| Buffer treatment control | 7.26 ± 1.54 | | | 2 | | 30 | |
| (n = 32) | | | | | | | |
| PlySs2 at 1.4 mg/kg (n = 24) | 8.24 ± 0.02 | 7 | | | | 17 | |
| PlySs2 at 0.7 mg/kg (n = 24) | 7.93 ± 0.12 | | | | 1 | 23 | |
| PlySs2 at 0.35 mg/kg (n = 24) | 8.17 ± 0.58 | 6 | | | | 18 | |
| PlySs2 at 0.18 mg/kg (n = 24) | 8.65 ± 0.58 | | | | | 16 | |

As shown in the Table 3, the PlySs2 MICs remained stable at 1 μg/mL. As shown in Table 4, however, the PlySs2 exposure resulted in increased oxacillin susceptibility. See, e.g., an oxacillin MIC of ≤2 μg/mL for 7 samples after PlySs2 exposure at 1.4 mg/kg and 6 samples after PlySs2 exposure at 0.35 mg/kg.

Table 5 shows the Log₁₀ CFU/g of the bacteria isolates on the valvular vegetation subject to the selective conditions, and Table 6 shows the MICs calculated for PlySs2 and oxacillin for valvular vegetations subject to the selective conditions.

TABLE 5
Log₁₀ CFU/g of Vegetation

| | Log₁₀ CFU/g of Vegetation | | | | |
|---|---|---|---|---|---|
| Treatment Group | 0 | 16 | 32 | 64 | 128 |
| Pre-treatment | 7.02 ± 1.47 | 4.3 ± 2.0 | <2.3 ± 0.04 | <2.3 ± 0.04 | <2.3 ± 0.04 |
| control (n = 19) | | | | | |
| Buffer treatment | 7.26 ± 1.54 | 3.3 ± 1.1 | 4.9 ± 1.8 | 3.1 ± 1.6 | <2.1 ± 0.1 |
| control (n = 44) | | | | | |
| PlySs2 at 1.4 mg/kg | 8.24 ± 0.02 | 7.1 ± 0.06 | 5.9 ± 0.03 | 4.2 ± 1.7 | 2.8 ± 1.2 |
| (n = 24) | | | | | |
| PlySs2 at 0.7 mg/kg | 7.93 ± 0.12 | 6.7 ± 0.1 | 5.7 ± 1.1 | 3.8 ± 1.8 | <2.1 ± 0.1 |
| (n = 24) | | | | | |
| PlySs2 at 0.35 mg/kg | 8.17 ± 0.58 | 6.9 ± 0.2 | 6.4 ± 0.5 | 5.5 ± 0.6 | 3.4 ± 1.3 |
| (n = 24) | | | | | |
| PlySs2 at 0.18 mg/kg | 8.65 ± 0.58 | 7.1 ± 0.1 | 7.1 ± 0.1 | 6.6 ± 0.9 | 4.6 ± 0.7 |
| (n = 24) | | | | | |

TABLE 6
PlySs2 and Oxacillin MICs

| Treatment | PlySs2 MIC (μg/mL) | | | | | Oxacillin MIC (μg/mL) | | | | | |
|---|---|---|---|---|---|---|---|---|---|---|---|
| Group | 0.25 | 0.5 | 1 | 2 | 4 | <2 | 4 | 8 | 16 | 32 | 64 |

| | | | | | | |
|---|---|---|---|---|---|---|
| Pre-treatment | 1 | 18 | | | | 19 |
| control (n = 19) | | | | | | |
| Buffer | | 44 | | | | 44 |
| treatment | | | | | | |
| control (n = 44) | | | | | | |
| PlySs2 at 1.4 | | 20 | 4 | 3 | | 21 |
| mg/kg (n = 24) | | | | | | |
| PlySs2 at 0.7 | 2 | 19 | 3 | | 3 | 21 |
| mg/kg (n = 24) | | | | | | |
| PlySs2 at 0.35 | | 22 | 2 | 7 | | 17 |
| mg/kg (n = 24) | | | | | | |
| PlySs2 at 0.18 | | 14 | 2 | | | 16 |
| mg/kg (n = 24) | | | | | | |

As shown in Table 6, the PlySs2 MICs remained largely stable at 1 μg/mL and exhibited only 2-fold increases, while PlySs2 exposure resulted in increased oxacillin susceptibility. See, e.g., an oxacillin MIC of ≤2 μg/mL for 3 samples after PlySs2 exposure at 1.4 mg/kg and 7 samples after PlySs2 exposure at 0.35 mg/kg. This evidences a greater than 16-fold reduction in oxacillin MIC values, from 32 μg/mL to <2 μg/mL. The resensitization observed in vivo was therefore greatly enhanced over that observed in vitro. Moreover, MIC increases of only up to 2-fold were observed for PlySs2.

As with the isolates exhibiting resensitization phenotypes in the serial passage assay discussed above in Example 2, isolates from the rabbit IE study likewise underwent whole genome sequencing and additional genetic analysis to identify specific mutations of interest.

Two mutants from the valvular vegetations exhibiting 32-fold decreases in oxacillin MIC were identified, analyzed by whole genome sequencing and SNPs/INDELs, and and compared to three control isolates. The PlySs2 and oxacillin MICs of each mutant and control strain are shown below in Tables 7 and 8, wherein + indicates the presence of the mutation and—indicates an absence of the mutation.

TABLE 7
Control strains for mutations associated with PlySs2-
mediated reductions in oxacillin MICs in vivo

| | | | | | Control #1/#2/#3 | |
|---|---|---|---|---|---|---|
| | | | | | PlySs2 | OXA |
| | | | | Amino | (1 μg/mL)/ | (64 μg/mL)/ |
| Ref. | Overlapping | | | Acid | (2 μg/mL)/ | (32 μg/mL)/ |
| Position^{a} | Annotation^{b} | Ref. | Allele | Change | (2 μg/mL) | (64 μg/mL) |

| | | | | | |
|---|---|---|---|---|---|
| 2492859 | hlgCB (near) | T | C | | −/−/− |
| 1366472 | mprF | T | A | L291I | −/−/− |
| 704001 | graR | T | G | I158S | −/−/− |
| 34167 | rlmH | G | A | K159R | −/−/− |

| | | |
|---|---|---|
| SCCmec | ΔSCCmec | −/−/− |

^{a}Position in the reference genome of S. aureus MW2 (GenBank accession: NC_003923.1)
^{b}Annotated open reading frames overlapping computationally-predicted polymorphisms

TABLE 8
Mutant strains for mutations associated with PlySs2-
mediated reductions in oxacillin MICs in vivo

| | | | | Amino | Mutant #1 | | Mutant #2 | |
|---|---|---|---|---|---|---|---|---|
| Ref. | Overlapping | | | Acid | PlySs2 | OXA | PlySs2 | OXA |
| Position^{a} | Annotation^{b} | Ref. | Allele | Change | (2 μg/mL) | (1 μg/mL) | (1 μg/mL) | (1 μg/mL) |

| | | | | | | |
|---|---|---|---|---|---|---|
| 2492859 | hlgCB (near) | T | C | | + | + |
| 1366472 | mprF | T | A | L291I | + | − |
| 704001 | graR | T | G | I158S | − | + |
| 34167 | rlmH | G | A | K159R | + | + |

| | | | |
|---|---|---|---|
| SCCmec | ΔSCCmec | + | + |

^{a}Position in the reference genome of S. aureus MW2 (GenBank accession: NC_003923.1)
^{b}Annotated open reading frames overlapping computationally-predicted polymorphisms

From the examples herein, it is concluded that PlySs2 treatment resensitized MRSA to β-lactam antibiotics in in vitro and in vivo studies. Potent synergy with PlySs2 reduces β-lactam MICs to below breakpoints without adverse impact on anticipated susceptibility to PlySs2. Moreover, exposure to PlySs2 alone may select for mutations in cell wall biosynthetic genes or SCCmec that decreases oxacillin MICs. By restoring sensitivity of MRSA strains to β-lactam antibiotics, PlySs2 may be used to not only combat, but also reverse, antimicrobial resistance.

1. A method of resensitizing a Gram-positive bacterium in a subject to at least one β-lactam antibiotic, comprising co-administering to the subject the at least one β-lactam antibiotic and a lysin polypeptide, thereby resensitizing the Gram-positive bacterium in the subject to the at least one β-lactam antibiotic.

2. The method according to claim 1, wherein the Gram-positive bacterium is a Staphylococcus bacterium.

3. The method according to claim 1, wherein the Gram-positive bacterium is Staphylococcus aureus.

4. The method according to claim 1, wherein the Gram-positive bacterium is methicillin-resistant Staphylococcus aureus (MRSA).

5. The method according to claim 1, wherein the Gram-positive bacterium is vancomycin-resistant Staphylococcus aureus (VRSA).

6. The method according to claim 1, wherein the at least one β-lactam antibiotic is selected from the group consisting of oxacillin, nafcillin, and cefazolin.

7. The method according to claim 1, wherein the at least one β-lactam antibiotic is oxacillin.

8. The method according to claim 1, wherein the Gram-positive bacterium causes skin or soft tissue infection, bacteremia, endocarditis, bone infection, joint infection, and/or pneumonia.

9. The method according to claim 8, wherein the bone infection is osteomyelitis.

10. The method according to claim 1, wherein after administration of the lysin polypeptide, the at least one β-lactam antibiotic is effective at a dosage below its MIC dose to reduce the population, kill, inhibit the growth, and/or eradicate the Gram-positive bacterium.

11. The method according to claim 1, further comprising, after the co-administration step, a step of administering the at least one β-lactam antibiotic to the subject in an amount effective to reduce the population, kill, inhibit the growth, and/or eradicate the Gram-positive bacterium.

12. The method according to claim 1, wherein the lysin polypeptide is administered in a dose below its MIC dose.

13. The method according to claim 1, wherein the lysin polypeptide is administered in a single dose.

14. The method according to claim 1, wherein the lysin polypeptide comprises an amino acid sequence selected from the group consisting of SEQ ID NOs. 1-17 or variants thereof having at least 80% amino acid identity to SEQ ID NOs. 1-17 and lytic activity.

15. The method according to claim 1, wherein the lysin polypeptide comprises an amino acid sequence of SEQ ID NO: 1.

16. The method according to claim 1, wherein the lysin polypeptide comprises an amino acid sequence selected from the group consisting of SEQ ID NOs. 3-17.

17. The method according to claim 1, wherein the lysin polypeptide is administered substantially simultaneously with the at least one β-lactam antibiotic.

18. The method according to claim 1, wherein the lysin polypeptide is administered prior to administration of the at least one β-lactam antibiotic.

19. The method according to claim 18, wherein the lysin polypeptide is administered at least 24 hours prior to administration of the at least one β-lactam antibiotic.

20. A method of resensitizing a Gram-positive bacterium on a non-living surface to at least one β-lactam antibiotic, comprising co-administering to the non-living surface at least one β-lactam antibiotic and a lysin polypeptide, wherein the non-living surface is infected with a Gram-positive bacterium that is resistant to the at least one β-lactam antibiotic and wherein the co-administration step reduces the amount of Gram-positive bacterium on the non-living surface and resensitizes the Gram-positive bacterium to the at least one β-lactam antibiotic.

21. The method of claim 20, further comprising after the co-administering step, a step of administering the at least one β-lactam antibiotic to the non-living surface in an amount effective to reduce the population, kill, inhibit the growth, and/or eradicate the resensitized Gram-positive bacterium.

22. The method of claim 20, wherein the non-living surface is surface of a medical device.


SEQUENCE LISTING (17 sequences)

SEQ ID NO: 1 - protein, 245 aa, Streptococcus suis
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 2 - protein, 10 aa, Streptococcus suis
PPGTVAQSAP

SEQ ID NO: 3 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 4 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEHFDYDTVIEDVNGYVWVSYIGGSGKRNYVATGATKDGKRFGNAWGTFK

SEQ ID NO: 5 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEQFDYDTAIIDVNGYAWVSYIGGSGKRNYVATGATKDGKRFGNAWGTFK

SEQ ID NO: 6 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTVIIDVNGYVWVSYIGGSGKRNYVATGATKDGKRFGNAWGTFK

SEQ ID NO: 7 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGESFDYDTVIIDVNGYVWVSYIGGSGKRNYVATGATKDGKRFGNAWGTFK

SEQ ID NO: 8 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSNRETGTMTVTVDALNVRRAPNTSGEIVAVYKEGESFDYDTVIIDVNGYVWVSYIGGSGKRNYVATGATKDGKRFGNAWGTFK

SEQ ID NO: 9 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGEHFDYDTVIIDVNGYVWVSYIGGSGKRNYVATGATKDGKRFGNAWGTFK

SEQ ID NO: 10 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGESFDYDTAIIDVNGYAWVSYIGGSGKRNYVATGATKDGKRFGNAWGTFK

SEQ ID NO: 11 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSNRETGTMTVTVDALNVRRAPDTSGEIVAVYKEGESFDYDTEIIDVNGYVWVSYIGGSGKRNYVATGATKDGKRFGNAWGTFK

SEQ ID NO: 12 - protein, 245 aa, Artificial Sequence
  Description of Artificial Sequence Synthetic polypeptide
MTTVNEALNNVRAQVGSGVSVGNGECYALASWYEEMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 13 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 14 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 15 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTKIIDVNGYEWVSYIGGSGKRNYVATGATKDGKRFGNAWGTFK

SEQ ID NO: 16 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTKIIDVNGYEWVSYIGGSGKRNYVATGATKDGKRFGNAWGTFK

SEQ ID NO: 17 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIEDVNGYVWGSYIGGSGKRNYVATGATKDGKRFGNAWGTFK